SEQUENCE LISTING (3 sequences)

SEQ ID NO: 1 - protein, 133 aa, Homo sapiens
MGSSHHHHHHSSGLVPRGSHMLETEEEEEEGAGATLSRGPAFPGMGSEELRLASFYDWPLTAEVPPELLAAAGFFHTGHQDKVRCFFCYGGLQSWKRGDDPWTEHAKWFPGCQFLLRSKGQEYINNIHLTHSL

SEQ ID NO: 2 - protein, 89 aa, Homo sapiens
MQTHAARMRTFMYWPSSVPVQPEQLAAAGFYYVGRNDDVKCFSCDGGLRCWESGDDPWVEHAKWFPGCEFLIRMKGQEYINNIHLTHSL

SEQ ID NO: 3 - protein, 89 aa, Homo sapiens
MQTHAARFKTFFNWPSSVLVNPEQLAAAGFYYVGNSDDVKCFSCDGGLRCWESGDDPWVQHAKWFPGCEYLIRIKGQEYINNIHLTHSL

(19) United States
(12) Patent Application Publication
(10) Pub. No.: US 2011/0218211 A1
(43) Pub. Date: Sep. 8, 2011

(54) AZAINDOLE INHIBITORS OF IAP

(75) Inventors: Philippe Bergeron, Oakland, CA (US); Michael F.T. Koehler, Palo Alto, CA (US)

(73) Assignee: Genentech, Inc., South San Francisco, CA (US)

(21) Appl. No.: 13/058,363

(22) PCT Filed: Aug. 14, 2009
(86) PCT No.: PCT/US09/53889

§ 371 Date: Apr. 29, 2011

Related U.S. Application Data
(60) Provisional application No. 61/089,508, filed on Aug. 16, 2008.

(51) Int. Cl. A61K 31/519; C07D 487/04; C12N 5/07; A61P 35/00

(52) U.S. Cl. 514/265.1; 544/280; 435/375

(57) ABSTRACT

The invention provides novel inhibitors of IAP that are useful as therapeutic agents for treating malignancies where the compounds have the general formula I: I wherein X₁, X₂, Y, Z₁, Z₂, Z₃, Z₄, R₁, R₂, R₃, R₃′, R₄, R₄′, R₅, R₆, R₆′ and R₉ are as described herein.

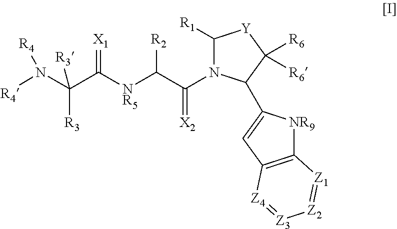

### PRIORITY CLAIM

This application claims priority to U.S. provisional application No. 61/089,508 filed on 16 Aug. 2008, the contents of which are incorporated herein by reference.

### FIELD OF THE INVENTION

The present invention relates to organic compounds useful for therapy and/or prophylaxis in a mammal, and in particular to inhibitors of IAP proteins useful for treating cancers.

### BACKGROUND OF THE INVENTION

Apoptosis or programmed cell death is a genetically and biochemically regulated mechanism that plays an important role in development and homeostasis in invertebrates as well as vertebrates. Aberrancies in apoptosis that lead to premature cell death have been linked to a variety of developmental disorders. Deficiencies in apoptosis that result in the lack of cell death have been linked to cancer and chronic viral infections (Thompson et al., (1995) Science 267, 1456-1462).

One of the key effector molecules in apoptosis are the caspases (cysteine containing aspartate specific proteases). Caspases are strong proteases, cleaving after aspartic acid residues and once activated, digest vital cell proteins from within the cell. Since caspases are such strong proteases, tight control of this family of proteins is necessary to prevent premature cell death. In general, caspases are synthesized as largely inactive zymogens that require proteolytic processing in order to be active. This proteolytic processing is only one of the ways in which caspases are regulated. The second mechanism is through a family of proteins that bind and inhibit caspases.

A family of molecules that inhibit caspases are the Inhibitors of Apoptosis (IAP) (Deveraux et al., J Clin Immunol (1999), 19:388-398). IAPs were originally discovered in baculovirus by their functional ability to substitute for P35 protein, an anti-apoptotic gene (Crook et al. (1993) J Virology 67, 2168-2174). IAPs have been described in organisms ranging from Drosophila to human. Regardless of their origin, structurally, IAPs comprise one to three Baculovirus IAP repeat (BIR) domains, and most of them also possess a carboxyl-terminal RING finger motif. The BIR domain itself is a zinc binding domain of about 70 residues comprising 4 alpha-helices and 3 beta strands, with cysteine and histidine residues that coordinate the zinc ion (Hinds et al., (1999) Nat. Struct. Biol. 6, 648-651). It is the BIR domain that is believed to cause the anti-apoptotic effect by inhibiting the caspases and thus inhibiting apoptosis. As an example, human X-chromosome linked IAP (XIAP) inhibits caspase 3, caspase 7 and the Apaf-1-cytochrome C mediated activation of caspase 9 (Deveraux et al., (1998) EMBO J. 17, 2215-2223). Caspases 3 and 7 are inhibited by the BIR2 domain of XIAP, while the BIR3 domain of XIAP is responsible for the inhibition of caspase 9 activity. XIAP is expressed ubiquitously in most adult and fetal tissues (Liston et al, Nature, 1996, 379(6563):349), and is overexpressed in a number of tumor cell lines of the NCI 60 cell line panel (Fong et al, Genomics, 2000, 70:113; Tamm et al, Clin. Cancer Res. 2000, 6(5):1796). Overexpression of XIAP in tumor cells has been demonstrated to confer protection against a variety of pro-apoptotic stimuli and promotes resistance to chemotherapy (LaCasse et al, Oncogene, 1998, 17(25):3247). Consistent with this, a strong correlation between XIAP protein levels and survival has been demonstrated for patients with acute myelogenous leukemia (Tamm et al, supra). Down-regulation of XIAP expression by antisense oligonucleotides has been shown to sensitize tumor cells to death induced by a wide range of pro-apoptotic agents, both in vitro and in vivo (Sasaki et al, Cancer Res., 2000, 60(20):5659; Lin et al, Biochem J., 2001, 353:299; Hu et al, Clin. Cancer Res., 2003, 9(7):2826). Smac/DIABLO-derived peptides have also been demonstrated to sensitize a number of different tumor cell lines to apoptosis induced by a variety of pro-apoptotic drugs (Arnt et al, J. Biol. Chem., 2002, 277(46):44236; Fulda et al, Nature Med., 2002, 8(8):808; Guo et al, Blood, 2002, 99(9):3419; Vucic et al, J. Biol. Chem., 2002, 277(14):12275; Yang et al, Cancer Res., 2003, 63(4):831).

Melanoma IAP (ML-IAP) is an IAP not detectable in most normal adult tissues but is strongly upregulated in melanoma (Vucic et al., (2000) Current Bio 10:1359-1366). Determination of protein structure demonstrated significant homology of the ML-IAP BIR and RING finger domains to corresponding domains present in human XIAP, cIAP1 and cIAP2. The BIR domain of ML-IAP appears to have the most similarities to the BIR2 and BIR3 of XIAP, cIAP1 and cIAP2, and appears to be responsible for the inhibition of apoptosis, as determined by deletional analysis. Furthermore, Vucic et al., demonstrated that ML-IAP could inhibit chemotherapeutic agent induced apoptosis. Agents such as adriamycin and 4-tertiary butylphenol (4-TBP) were tested in a cell culture system of melanomas overexpressing ML-IAP and the chemotherapeutic agents were significantly less effective in killing the cells when compared to a normal melanocyte control. The mechanism by which ML-IAP produces an anti-apoptotic activity is in part through inhibition of caspase 3 and 9. ML-IAP did not effectively inhibit caspases 1, 2, 6, or 8.

Since apoptosis is a strictly controlled pathway with multiple interacting factors, the discovery that IAPs themselves are regulated was not unusual. In the fruit fly Drosophila, the Reaper (rpr), Head Involution Defective (hid) and GRIM proteins physically interact with and inhibit the anti-apoptotic activity of the Drosophila family of IAPs. In the mammal, the proteins SMAC/DIABLO act to block the IAPs and allow apoptosis to proceed. It was shown that during normal apoptosis, SMAC is processed into an active form and is released from the mitochondria into the cytoplasm where it physically binds to IAPs and prevents the IAP from binding to a caspase. This inhibition of the IAP allows the caspase to remain active and thus proceed with apoptosis. Interestingly, sequence homology between the IAP inhibitors shows that there is a four amino acid motif in the N-terminus of the processed, active proteins. This tetrapeptide appears to bind into a hydrophobic pocket in the BIR domain and disrupts the BIR domain binding to caspases (Chai et al., (2000) Nature 406:855-862, Liu et al., (2000) Nature 408:1004-1008, Wu et al., (2000) Nature 408 1008-1012).

### SUMMARY OF THE INVENTION

In one aspect of the present invention there is provided novel inhibitors of IAP proteins having the general formula (I)

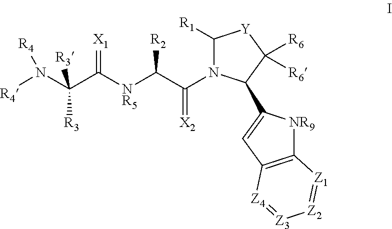

wherein
X₁ and X₂ are each independently O or S;
Y is a bond, (CR₇R₇)ₘ, O or S;
Z₁, Z₂, Z₃ and Z₄ are each independently N or CQ;
Q is H, halogen, hydroxyl, carboxyl, amino, nitro, cyano, alkyl, a carbocycle or a heterocycle; wherein said alkyl, carbocycle and heterocycle is optionally substituted with one or more hydroxyl, alkoxy, acyl, halogen, mercapto, oxo, carboxyl, acyl, optionally substituted alkyl, amino, cyano, nitro, amidino, guanidino an optionally substituted carbocycle or an optionally substituted heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂⁻NR₈—, —NR₈—SO₂—, —NR₈—C(O)—NR₈—, —NR₈—C(NH)—NR₈—, —NR₈—C(NH)—, —C(O)—O— or —O—C(O)—;
R₁ is H, OH or alkyl; or R₁ and R₂ together form a 5-8 member heterocycle;
R₂ is alkyl, a carbocycle, carbocyclylalkyl, a heterocycle or heterocyclylalkyl each optionally substituted with halogen, hydroxyl, oxo, thione, mercapto, carboxyl, alkyl, haloalkyl, acyl, alkoxy, alkylthio, sulfonyl, amino and nitro, wherein said alkyl, acyl, alkoxy, alkylthio and sulfonyl are optionally substituted with hydroxy, mercapto, halogen, amino, alkoxy, hydroxyalkoxy and alkoxyalkoxy;
R₃ is H or alkyl optionally substituted with halogen or hydroxyl; or R₃ and R₄ together form a 3-6 heterocycle;
R₃′ is H, or R₃ and R₃′ together form a 3-6 carbocycle;
R₄ and R₄′ are independently H, hydroxyl, amino, alkyl, carbocycle, carbocycloalkyl, carbocycloalkyloxy, carbocycloalkyloxycarbonyl, heterocycle, heterocycloalkyl, heterocycloalkyloxy or heterocycloalkyloxycarbonyl; wherein each alkyl, carbocycloalkyl, carbocycloalkyloxy, carbocycloalkyloxycarbonyl, heterocycle, heterocycloalkyl, heterocycloalkyloxy and heterocycloalkyloxycarbonyl is optionally substituted with halogen, hydroxyl, mercapto, carboxyl, alkyl, alkoxy, amino, imino and nitro; or R₄ and R₄′ together form a heterocycle;
R₅ is H or alkyl;
R₆, and R₆′ are each independently H, alkyl, aryl or aralkyl;
R₇ is H, cyano, hydroxyl, mercapto, halogen, nitro, carboxyl, amidino, guanidino, alkyl, a carbocycle, a heterocycle or —U-V; wherein U is —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)— and V is alkyl, a carbocycle or a heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —C(O)—O— or —O—C(O)—; and an alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle;
R₈ is H, alkyl, a carbocycle or a heterocycle wherein one or more CH₂ or CH groups of said alkyl is optionally replaced with —O—, —S—, —S(O)—, S(O)₂, —N(R₈), or —C(O)—; and said alkyl, carbocycle and heterocycle is optionally substituted with hydroxyl, alkoxy, acyl, halogen, mercapto, oxo (═O), carboxyl, acyl, halo-substituted alkyl, amino, cyano nitro, amidino, guanidino an optionally substituted carbocycle or an optionally substituted heterocycle;
R₉ is H, alkyl or acyl; and
m is 0 to 4.

In another aspect of the invention, there are provided compositions comprising compounds of formula I and a carrier, diluent or excipient.

In another aspect of the invention, there is provided a method of inducing apoptosis in a cell comprising introducing into said cell a compound of formula I.

In another aspect of the invention, there is provided a method of sensitizing a cell to an apoptotic signal comprising introducing into said cell a compound of formula I.

In another aspect of the invention, there is provided a method for inhibiting the binding of an IAP protein to a caspase protein comprising contacting said IAP protein with a compound of formula I.

In another aspect of the invention, there is provided a method for treating a disease or condition associated with the overexpression of an IAP protein in a mammal, comprising administering to said mammal an effective amount of a compound of formula I.

### DETAILED DESCRIPTION OF THE PREFERRED EMBODIMENTS

“Acyl” means a carbonyl containing substituent represented by the formula —C(O)—R in which R is H, alkyl, a carbocycle, a heterocycle, carbocycle-substituted alkyl or heterocycle-substituted alkyl wherein the alkyl, alkoxy, carbocycle and heterocycle are as defined herein. Acyl groups include alkanoyl (e.g. acetyl), aroyl (e.g. benzoyl), and heteroaroyl.

“Alkyl” means a branched or unbranched, saturated or unsaturated (i.e. alkenyl, alkynyl) aliphatic hydrocarbon group, having up to 12 carbon atoms unless otherwise specified. When used as part of another term, for example “alkylamino”, the alkyl portion may be a saturated hydrocarbon chain, however also includes unsaturated hydrocarbon carbon chains such as “alkenylamino” and “alkynylamino. Examples of particular alkyl groups are methyl, ethyl, n-propyl, isopropyl, n-butyl, iso-butyl, sec-butyl, tert-butyl, n-pentyl, 2-methylbutyl, 2,2-dimethylpropyl, n-hexyl, 2-methylpentyl, 2,2-dimethylbutyl, n-heptyl, 3-heptyl, 2-methylhexyl, and the like. The terms “lower alkyl” “C₁-C₄ alkyl” and “alkyl of 1 to 4 carbon atoms” are synonymous and used interchangeably to mean methyl, ethyl, 1-propyl, isopropyl, cyclopropyl, 1-butyl, sec-butyl or t-butyl. Unless specified, substituted, alkyl groups may contain one, for example two, three or four substituents which may be the same or different. Examples of substituents are, unless otherwise defined, halogen, amino, hydroxyl, protected hydroxyl, mercapto, carboxy, alkoxy, nitro, cyano, amidino, guanidino, urea, sulfonyl, sulfinyl, aminosulfonyl, alkylsulfonylamino, arylsulfonylamino, aminocarbonyl, acylamino, alkoxy, acyl, acyloxy, a carbocycle, a heterocycle. Examples of the above substituted alkyl groups include, but are not limited to; cyanomethyl, nitromethyl, hydroxymethyl, trityloxymethyl, propionyloxymethyl, aminomethyl, carboxymethyl, carboxyethyl, carboxypropyl, alkyloxycarbonylmethyl, allyloxycarbonylaminomethyl, carbamoyloxymethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichloro(n-butyl), 2-amino(iso-propyl), 2-carbamoyloxyethyl and the like. The alkyl group may also be substituted with a carbocycle group. Examples include cyclopropylmethyl, cyclobutylmethyl, cyclopentylmethyl, and cyclohexylmethyl groups, as well as the corresponding -ethyl, -propyl, -butyl, -pentyl, -hexyl groups, etc. Substituted alkyls include substituted methyls e.g. a methyl group substituted by the same substituents as the “substituted Cₙ—Cₘ alkyl” group. Examples of the substituted methyl group include groups such as hydroxymethyl, protected hydroxymethyl (e.g. tetrahydropyranyloxymethyl), acetoxymethyl, carbamoyloxymethyl, trifluoromethyl, chloromethyl, carboxymethyl, bromomethyl and iodomethyl.

“Amidine” means the group —C(NH)—NHR in which R is H, alkyl, a carbocycle, a heterocycle, carbocycle-substituted alkyl or heterocycle-substituted alkyl wherein the alkyl, alkoxy, carbocycle and heterocycle are as defined herein. A particular amidine is the group —NH—C(NH)—NH₂.

“Amino” means primary (i.e. —NH₂), secondary (i.e. —NRH) and tertiary (i.e. —NRR) amines in which R is H, alkyl, a carbocycle, a heterocycle, carbocycle-substituted alkyl or heterocycle-substituted alkyl wherein the alkyl, alkoxy, carbocycle and heterocycle are as defined herein. Particular secondary and tertiary amines are alkylamine, dialkylamine, arylamine, diarylamine, aralkylamine and diaralkylamine wherein the alkyl is as herein defined and optionally substituted. Particular secondary and tertiary amines are methylamine, ethylamine, propylamine, isopropylamine, phenylamine, benzylamine dimethylamine, diethylamine, dipropylamine and diisopropylamine.

“Amino-protecting group” as used herein refers to a derivative of the groups commonly employed to block or protect an amino group while reactions are carried out on other functional groups on the compound. Examples of such protecting groups include carbamates, amides, alkyl and aryl groups, imines, as well as many N-heteroatom derivatives which can be removed to regenerate the desired amine group. Particular amino protecting groups are Boc, Fmoc and Cbz. Further examples of these groups are found in T. W. Greene and P. G. M. Wuts, “Protective Groups in Organic Synthesis”, 2^{nd} ed., John Wiley & Sons, Inc., New York, N.Y., 1991, chapter 7; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected amino” refers to an amino group substituted with one of the above amino-protecting groups.

“Aryl” when used alone or as part of another term means a carbocyclic aromatic group whether or not fused having the number of carbon atoms designated or if no number is designated, up to 14 carbon atoms. Particular aryl groups are phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). A particular aryl is phenyl. Substituted phenyl or substituted aryl means a phenyl group or aryl group substituted with one, two, three, four or five, for example 1-2, 1-3 or 1-4 substituents chosen, unless otherwise specified, from halogen (F, Cl, Br, I), hydroxy, protected hydroxy, cyano, nitro, alkyl (for example C₁-C₆ alkyl), alkoxy (for example C₁-C₆ alkoxy), benzyloxy, carboxy, protected carboxy, carboxymethyl, protected carboxymethyl, hydroxymethyl, protected hydroxymethyl, aminomethyl, protected aminomethyl, trifluoromethyl, alkylsulfonylamino, alkylsulfonylamino alkyl, arylsulfonylamino, arylsulonylamino alkyl, heterocyclylsulfonylamino, heterocyclylsulfonylamino alkyl, heterocyclyl, aryl, or other groups specified. One or more methyne (CH) and/or methylene (CH₂) groups in these substituents may in turn be substituted with a similar group as those denoted above. Examples of the term “substituted phenyl” includes but is not limited to a mono- or di(halo)phenyl group such as 2-chlorophenyl, 2-bromophenyl, 4-chlorophenyl, 2,6-dichlorophenyl, 2,5-dichlorophenyl, 3,4-dichlorophenyl, 3-chlorophenyl, 3-bromophenyl, 4-bromophenyl, 3,4-dibromophenyl, 3-chloro-4-fluorophenyl, 2-fluorophenyl and the like; a mono- or di(hydroxy)phenyl group such as 4-hydroxyphenyl, 3-hydroxyphenyl, 2,4-dihydroxyphenyl, the protected-hydroxy derivatives thereof and the like; a nitrophenyl group such as 3- or 4-nitrophenyl; a cyanophenyl group, for example, 4-cyanophenyl; a mono- or di(lower alkyl)phenyl group such as 4-methylphenyl, 2,4-dimethylphenyl, 2-methylphenyl, 4-(iso-propyl)phenyl, 4-ethylphenyl, 3-(n-propyl)phenyl and the like; a mono or di(alkoxy)phenyl group, for example, 3,4-dimethoxyphenyl, 3-methoxy-4-benzyloxyphenyl, 3-methoxy-4-(1-chloromethyl)benzyloxy-phenyl, 3-ethoxyphenyl, 4-(isopropoxy)phenyl, 4-(t-butoxy)phenyl, 3-ethoxy-4-methoxyphenyl and the like; 3- or 4-trifluoromethylphenyl; a mono- or dicarboxyphenyl or (protected carboxy)phenyl group such 4-carboxyphenyl; a mono- or di(hydroxymethyl)phenyl or (protected hydroxymethyl)phenyl such as 3-(protected hydroxymethyl)phenyl or 3,4-di(hydroxymethyl)phenyl; a mono- or di(aminomethyl)phenyl or (protected aminomethyl)phenyl such as 2-(aminomethyl)phenyl or 2,4-(protected aminomethyl)phenyl; or a mono- or di(N-(methylsulfonylamino))phenyl such as 3-(N-methylsulfonylamino))phenyl. Also, the term “substituted phenyl” represents disubstituted phenyl groups where the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy-4-chlorophenyl, and the like, as well as trisubstituted phenyl groups where the substituents are different, for example 3-methoxy-4-benzyloxy-6-methyl sulfonylamino, 3-methoxy-4-benzyloxy-6-phenyl sulfonylamino, and tetrasubstituted phenyl groups where the substituents are different such as 3-methoxy-4-benzyloxy-5-methyl-6-phenyl sulfonylamino Particular substituted phenyl groups include the 2-chlorophenyl, 2-aminophenyl, 2-bromophenyl, 3-methoxyphenyl, 3-ethoxy-phenyl, 4-benzyloxyphenyl, 4-methoxyphenyl, 3-ethoxy-4-benzyloxyphenyl, 3,4-diethoxyphenyl, 3-methoxy-4-benzyloxyphenyl, 3-methoxy-4-(1-chloromethyl)benzyloxy-phenyl, 3-methoxy-4-(1-chloromethyl)benzyloxy-6-methyl sulfonyl aminophenyl groups. Fused aryl rings may also be substituted with any, for example 1, 2 or 3, of the substituents specified herein in the same manner as substituted alkyl groups.

“Carbocyclyl”, “carbocyclylic”, “carbocycle” and “carbocyclo” alone and when used as a moiety in a complex group such as a carbocycloalkyl group, refers to a mono-, bi-, or tricyclic aliphatic ring having 3 to 14 carbon atoms, for example 3 to 7 carbon atoms, which may be saturated or unsaturated, aromatic or non-aromatic. Particular saturated carbocyclic groups are cyclopropyl, cyclobutyl, cyclopentyl and cyclohexyl groups. A particular saturated carbocycle is cyclopropyl. Another particular saturated carbocycle is cyclohexyl. Particular unsaturated carbocycles are aromatic e.g. aryl groups as previously defined, for example phenyl. The terms “substituted carbocyclyl”, “carbocycle” and “carbocyclo” mean these groups substituted by the same substituents as the “substituted alkyl” group.

“Carboxy-protecting group” as used herein refers to one of the ester derivatives of the carboxylic acid group commonly employed to block or protect the carboxylic acid group while reactions are carried out on other functional groups on the compound. Examples of such carboxylic acid protecting groups include 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl such as t-butyl or t-amyl, trityl, 4-methoxytrityl, 4,4′-dimethoxytrityl, 4,4′,4″-trimethoxytrityl, 2-phenylprop-2-yl, trimethylsilyl, t-butyldimethylsilyl, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. The species of carboxy-protecting group employed is not critical so long as the derivatized carboxylic acid is stable to the condition of subsequent reaction(s) on other positions of the molecule and can be removed at the appropriate point without disrupting the remainder of the molecule. In particular, it is important not to subject a carboxy-protected molecule to strong nucleophilic bases, such as lithium hydroxide or NaOH, or reductive conditions employing highly activated metal hydrides such as LiAlH₄. (Such harsh removal conditions are also to be avoided when removing amino-protecting groups and hydroxy-protecting groups, discussed below.) Particular carboxylic acid protecting groups are the alkyl (e.g. methyl, ethyl, t-butyl), allyl, benzyl and p-nitrobenzyl groups. Similar carboxy-protecting groups used in the cephalosporin, penicillin and peptide arts can also be used to protect a carboxy group substituents. Further examples of these groups are found in T. W. Greene and P. G. M. Wuts, “Protective Groups in Organic Synthesis”, 2^{nd} ed., John Wiley & Sons, Inc., New York, N.Y., 1991, chapter 5; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981, Chapter 5. The term “protected carboxy” refers to a carboxy group substituted with one of the above carboxy-protecting groups.

“Guanidine” means the group —NH—C(NH)—NHR in which R is H, alkyl, a carbocycle, a heterocycle, carbocycle-substituted alkyl or heterocycle-substituted alkyl wherein the alkyl, alkoxy, carbocycle and heterocycle are as defined herein. A particular guanidine is the group —NH—C(NH)—NH₂.

“Hydroxy-protecting group” as used herein refers to a derivative of the hydroxy group commonly employed to block or protect the hydroxy group while reactions are carried out on other functional groups on the compound. Examples of such protecting groups include tetrahydropyranyloxy, benzoyl, acetoxy, carbamoyloxy, benzyl, and silylethers (e.g. TBS, TBDPS) groups. Further examples of these groups are found in T. W. Greene and P. G. M. Wuts, “Protective Groups in Organic Synthesis”, 2^{nd} ed., John Wiley & Sons, Inc., New York, N.Y., 1991, chapters 2-3; E. Haslam, “Protective Groups in Organic Chemistry”, J. G. W. McOmie, Ed., Plenum Press, New York, N.Y., 1973, Chapter 5, and T. W. Greene, “Protective Groups in Organic Synthesis”, John Wiley and Sons, New York, N.Y., 1981. The term “protected hydroxy” refers to a hydroxy group substituted with one of the above hydroxy-protecting groups.

“Heterocyclic group”, “heterocyclic”, “heterocycle”, “heterocyclyl”, or “heterocyclo” alone and when used as a moiety in a complex group such as a heterocycloalkyl group, are used interchangeably and refer to any mono-, bi-, or tricyclic, saturated or unsaturated, aromatic (heteroaryl) or non-aromatic ring having the number of atoms designated, generally from 5 to about 14 ring atoms, where the ring atoms are carbon and at least one heteroatom (nitrogen, sulfur or oxygen), for example 1 to 4 heteroatoms. Typically, a 5-membered ring has 0 to 2 double bonds and 6- or 7-membered ring has 0 to 3 double bonds and the nitrogen or sulfur heteroatoms may optionally be oxidized (e.g. SO, SO₂), and any nitrogen heteroatom may optionally be quaternized. Particular non-aromatic heterocycles are morpholinyl (morpholino), pyrrolidinyl, oxiranyl, oxetanyl, tetrahydrofuranyl, 2,3-dihydrofuranyl, 2H-pyranyl, tetrahydropyranyl, thiiranyl, thietanyl, tetrahydrothietanyl, aziridinyl, azetidinyl, 1-methyl-2-pyrrolyl, piperazinyl and piperidinyl. A “heterocycloalkyl” group is a heterocycle group as defined above covalently bonded to an alkyl group as defined above. Particular 5-membered heterocycles containing a sulfur or oxygen atom and one to three nitrogen atoms are thiazolyl, in particular thiazol-2-yl and thiazol-2-yl N-oxide, thiadiazolyl, in particular 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Particular 5-membered ring heterocycles containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Particular benzo-fused 5-membered heterocycles are benzoxazol-2-yl, benzthiazol-2-yl and benzimidazol-2-yl. Particular 6-membered heterocycles contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are a particular group. Substituents for “optionally substituted heterocycles”, and further examples of the 5- and 6-membered ring systems discussed above can be found in W. Druckheimer et al., U.S. Pat. No. 4,278,793. In a particular embodiment, such optionally substittuted heterocycle groups are substituted with hydroxyl, alkyl, alkoxy, acyl, halogen, mercapto, oxo, carboxyl, acyl, halo-substituted alkyl, amino, cyano, nitro, amidino and guanidino.

“Heteroaryl” alone and when used as a moiety in a complex group such as a heteroaralkyl group, refers to any mono-, bi-, or tricyclic aromatic ring system having the number of atoms designated where at least one ring is a 5-, 6- or 7-membered ring containing from one to four heteroatoms selected from the group nitrogen, oxygen, and sulfur, and in a particular embodiment at least one heteroatom is nitrogen (Lang's Handbook of Chemistry, supra). Included in the definition are any bicyclic groups where any of the above heteroaryl rings are fused to a benzene ring. Particular heteroaryls incorporate a nitrogen or oxygen heteroatom. The following ring systems are examples of the heteroaryl (whether substituted or unsubstituted) groups denoted by the term “heteroaryl”: thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, thiazinyl, oxazinyl, triazinyl, thiadiazinyl, oxadiazinyl, dithiazinyl, dioxazinyl, oxathiazinyl, tetrazinyl, thiatriazinyl, oxatriazinyl, dithiadiazinyl, imidazolinyl, dihydropyrimidyl, tetrahydropyrimidyl, tetrazolo[1,5-b]pyridazinyl and purinyl, as well as benzo-fused derivatives, for example benzoxazolyl, benzofuryl, benzothiazolyl, benzothiadiazolyl, benzotriazolyl, benzoimidazolyl and indolyl. A particular “heteroaryl” is: 1,3-thiazol-2-yl, 4-(carboxymethyl)-5-methyl-1,3-thiazol-2-yl, 4-(carboxymethyl)-5-methyl-1,3-thiazol-2-yl sodium salt, 1,2,4-thiadiazol-5-yl, 3-methyl-1,2,4-thiadiazol-5-yl, 1,3,4-triazol-5-yl, 2-methyl-1,3,4-triazol-5-yl, 2-hydroxy-1,3,4-triazol-5-yl, 2-carboxy-4-methyl-1,3,4-triazol-5-yl sodium salt, 2-carboxy-4-methyl-1,3,4-triazol-5-yl, 1,3-oxazol-2-yl, 1,3,4-oxadiazol-5-yl, 2-methyl-1,3,4-oxadiazol-5-yl, 2-(hydroxymethyl)-1,3,4-oxadiazol-5-yl, 1,2,4-oxadiazol-5-yl, 1,3,4-thiadiazol-5-yl, 2-thiol-1,3,4-thiadiazol-5-yl, 2-(methylthio)-1,3,4-thiadiazol-5-yl, 2-amino-1,3,4-thiadiazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 2-methyl-1H-tetrazol-5-yl, 1,2,3-triazol-5-yl, 1-methyl-1,2,3-triazol-5-yl, 2-methyl-1,2,3-triazol-5-yl, 4-methyl-1,2,3-triazol-5-yl, pyrid-2-yl N-oxide, 6-methoxy-2-(n-oxide)-pyridaz-3-yl, 6-hydroxypyridaz-3-yl, 1-methylpyrid-2-yl, 1-methylpyrid-4-yl, 2-hydroxypyrimid-4-yl, 1,4,5,6-tetrahydro-5,6-dioxo-4-methyl-as-triazin-3-yl, 1,4,5,6-tetrahydro-4-(formylmethyl)-5,6-dioxo-as-triazin-3-yl, 2,5-dihydro-5-oxo-6-hydroxy-astriazin-3-yl, 2,5-dihydro-5-oxo-6-hydroxy-as-triazin-3-yl sodium salt, 2,5-dihydro-5-oxo-6-hydroxy-2-methyl-astriazin-3-yl sodium salt, 2,5-dihydro-5-oxo-6-hydroxy-2-methyl-as-triazin-3-yl, 2,5-dihydro-5-oxo-6-methoxy-2-methyl-as-triazin-3-yl, 2,5-dihydro-5-oxo-as-triazin-3-yl, 2,5-dihydro-5-oxo-2-methyl-as-triazin-3-yl, 2,5-dihydro-5-oxo-2,6-dimethyl-as-triazin-3-yl, tetrazolo[1,5-b]pyridazin-6-yl and 8-aminotetrazolo[1,5-b]-pyridazin-6-yl. An alternative group of “heteroaryl” includes; 4-(carboxymethyl)-5-methyl-1,3-thiazol-2-yl, 4-(carboxymethyl)-5-methyl-1,3-thiazol-2-yl sodium salt, 1,3,4-triazol-5-yl, 2-methyl-1,3,4-triazol-5-yl, 1H-tetrazol-5-yl, 1-methyl-1H-tetrazol-5-yl, 1-(1-(dimethylamino)eth-2-yl)-1H-tetrazol-5-yl, 1-(carboxymethyl)-1H-tetrazol-5-yl, 1-(carboxymethyl)-1H-tetrazol-5-yl sodium salt, 1-(methylsulfonic acid)-1H-tetrazol-5-yl, 1-(methylsulfonic acid)-1H-tetrazol-5-yl sodium salt, 1,2,3-triazol-5-yl, 1,4,5,6-tetrahydro-5,6-dioxo-4-methyl-as-triazin-3-yl, 1,4,5,6-tetrahydro-4-(2-formylmethyl)-5,6-dioxo-as-triazin-3-yl, 2,5-dihydro-5-oxo-6-hydroxy-2-methyl-as-triazin-3-yl sodium salt, 2,5-dihydro-5-oxo-6-hydroxy-2-methyl-as-triazin-3-yl, tetrazolo[1,5-b]pyridazin-6-yl, and 8-aminotetrazolo[1,5-b]pyridazin-6-yl. Heteroaryl groups are optionally substituted as described for heterocycles.

“Inhibitor” means a compound which reduces or prevents the binding of IAP proteins to caspase proteins or which reduces or prevents the inhibition of apoptosis by an IAP protein. Alternatively, “inhibitor” means a compound which prevents the binding interaction of XIAP with caspases or the binding interaction of ML-IAP with SMAC.

“Optionally substituted” unless otherwise specified means that a group may be unsubstituted or substituted by one or more (e.g. 0, 1, 2, 3 or 4) of the substituents listed for that group in which said substituents may be the same or different. In an embodiment an optionally substituted group has 1 substituent. In another embodiment an optionally substituted group has 2 substituents. In another embodiment an optionally substituted group has 3 substituents.

“Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

“Pharmaceutically acceptable base addition salts” include those derived from inorganic bases such as sodium, potassium, lithium, ammonium, calcium, magnesium, iron, zinc, copper, manganese, aluminum salts and the like. Particularly base addition salts are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly organic non-toxic bases are isopropylamine, diethylamine, ethanolamine, trimethamine, dicyclohexylamine, choline, and caffeine.

“Sulfonyl” means a —SO₂—R group in which R is H, alkyl, a carbocycle, a heterocycle, carbocycle-substituted alkyl or heterocycle-substituted alkyl wherein the alkyl, alkoxy, carbocycle and heterocycle are as defined herein. Particular sulfonyl groups are alkylsulfonyl (i.e. —SO₂-alkyl), for example methylsulfonyl; arylsulfonyl, for example phenylsulfonyl; aralkylsulfonyl, for example benzylsulfonyl.

The phrase “and salts and solvates thereof” as used herein means that compounds of the inventions may exist in one or a mixture of salts and solvate forms. For example a compound of the invention may be substantially pure in one particular salt or solvate form or else may be mixtures of two or more salt or solvate forms.

The present invention provides novel compounds having the general formula I:

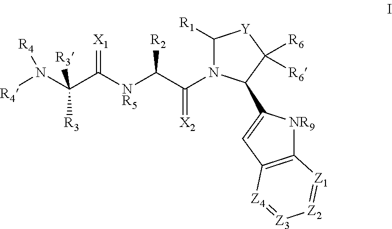

wherein X₁, X₂, Y, Z₁, Z₂, Z₃, Z₄, R₁, R₂, R₃, R₃′, R₄, R₄′, R₅, R₆, R₆′ and R₉ are as described herein provided that the compound is other than: 2-[(2R)-1-[N-methyl-L-alanyl-(25)-2-cyclohexylglycyl]-2-pyrrolidinyl]-1H-indole. Compounds of the invention include salts, solvates and polymorphs thereof unless otherwise specified.

X₁ and X₂ are each independently O or S. In a particular embodiment, X₁ and X₂ are both O. In another particular embodiment X₁ and X₂ are both S. In another particular embodiment, X₁ is S while X₂ is O. In another particular embodiment, X₁ is O while X₂ is S.

Y is a bond, (CR₇R₇)ₘ, O or S. In an embodiment Y is a bond, (CR₇R₇)ₘ, O or S; wherein m is 1 or 2 and R₇ is as defined herein or is H, halogen, alkyl, aryl, aralkyl, amino, arylamino, alkylamino, aralkylamino, alkoxy, aryloxy or aralkyloxy. In a particular embodiment, Y is (CHR₇)ₘ, O or S; wherein m is 1 or 2 and R₇ is H, halogen, alkyl, aryl, aralkyl, amino, arylamino, alkylamino, aralkylamino, alkoxy, aryloxy or aralkyloxy. In a particular embodiment, Y is CH₂. In a particular embodiment m is 1. In a particular embodiment Y is a bond. In a particular embodiment m is 1 and Y is CHR₇ wherein R₇ is aralkyloxy, for example benzyloxy. In a particular embodiment m is 1 and Y is CHR₇ wherein R₇ is F. In a particular embodiment m is 1 and Y is CHR₇ wherein R₇ is aralkylamino, for example benzylamino. In another particular embodiment Y is O. In another particular embodiment Y is S.

Z₁, Z₂, Z₃ and Z₄ are each independently N or CQ. In an embodiment Z₁ is N. In an embodiment Z₁ is CQ. In an embodiment Z₂ is N. In an embodiment Z₂ is CQ. In an embodiment Z₃ is N. In an embodiment Z₃ is CQ. In an embodiment Z₄ is N. In an embodiment Z₄ is CQ. In an embodiment Z₁ is N. In an embodiment Z₁ is Q.

In an embodiment Z₁, Z₂, Z₃ and Z₄ are each independently CQ. In an embodiment Z₁, Z₂ and Z₃ are each CQ and Z₄ is N. In an embodiment Z₁, Z₂ and Z₄ are each independently CQ and Z₃ is N. In an embodiment Z₁, Z₃ and Z₄ are each independently CQ and Z₂ is N. In an embodiment Z₂, Z₃ and Z₄ are each independently CQ and Z₁ is N. In an embodiment Z₁ and Z₃ are each independently CQ and Z₂ and Z₄ are each N.

Q is H, halogen, hydroxyl, carboxyl, amino, nitro, cyano, alkyl, a carbocycle or a heterocycle; wherein said alkyl, carbocycle and heterocycle is optionally substituted with one or more hydroxyl, alkoxy, acyl, halogen, mercapto, oxo, carboxyl, acyl, optionally substituted alkyl, amino, cyano, nitro, amidino, guanidino an optionally substituted carbocycle or an optionally substituted heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)—. Substituents of the “optionally substituted alkyl”, “optionally substituted carbocycle” and “optionally substituted heterocycle” are substituted as the foregoing alkyl, carbocycle and heterocycle groups in Q. In a particular embodiment substituents of such “optionally substituted alkyl” are hydroxyl, alkoxy, acyl, halogen, mercapto, oxo, carboxyl, acyl, amino, cyano, nitro, amidino and guanidino. In a particular embodiment such optionally substituted carbocycle and heterocycle groups are substituted with hydroxyl, alkyl, alkoxy, acyl, halogen, mercapto, oxo, carboxyl, acyl, halo-substituted alkyl, amino, cyano, nitro, amidino and guanidino. In a particular embodiment Q is a carbocycle or heterocycle optionally substituted with halogen, amino, oxo, alkyl, a carbocycle or a heterocycle; wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)—; and wherein said alkyl, carbocycle or heterocycle is optionally substituted with halogen, amino, hydroxyl, mercapto, carboxyl, alkoxy, alkoxyalkoxy, hydroxyalkoxy, alkylthio, acyloxy, acyloxyalkoxy, alkylsulfonyl, alkylsulfonylalkyl, alkylsulfinyl, and alkylsulfinylalkyl. In a particular embodiment, only one instance of Q is other than H. In a particular embodiment, two or less instances of Q are other than H. In a particular embodiment, only one instance of Q is other than H and is a group of formula III-1 to III-16 or IIIa to IIIs. In a particular embodiment, two instances of Q are other than H wherein only one is a group of formula III-1 to III-16 or IIIa to IIIs.

In a particular embodiment, Q is a carbocycle or heterocycle selected from the group consisting of III-1-III-16 or IIIa to IIIs,

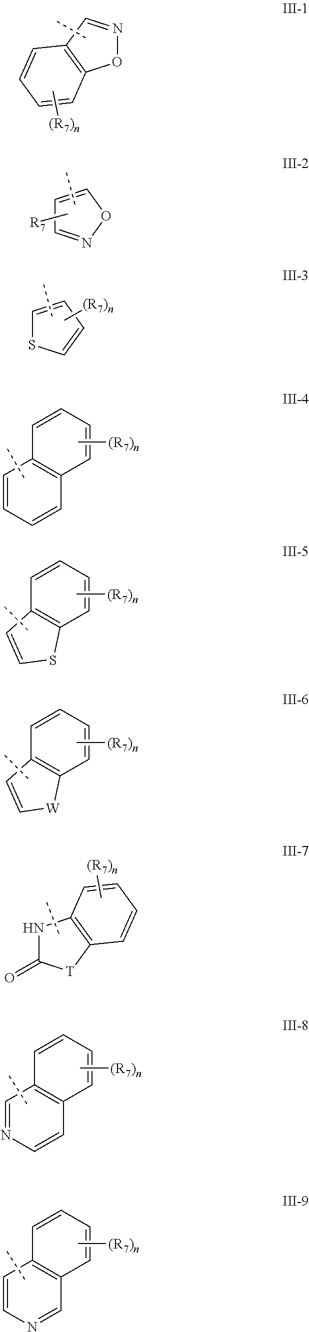

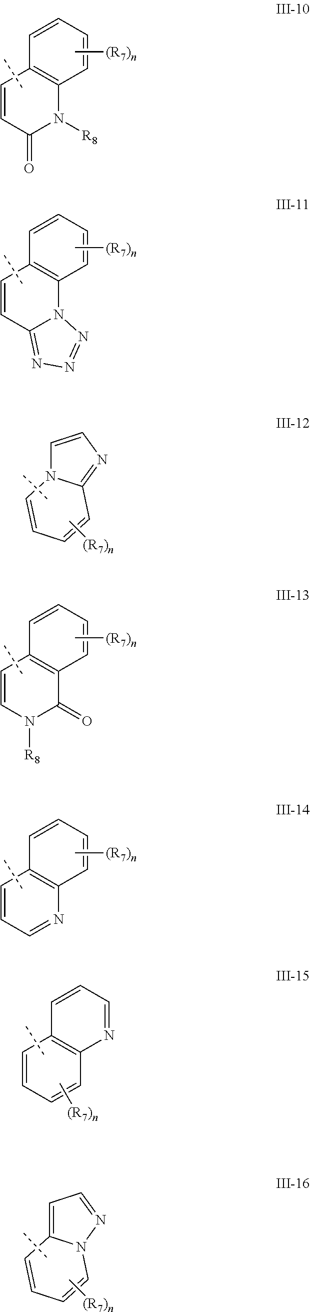

wherein n is 1-4, for example 1-3, for example 1-2, for example 1; T is O, S, NR₈ or CR₇R₇; W is O, NR₈ or CR₇R₇; and R₇ and R₈ are as defined herein. In an embodiment, one Q is a group of the general formula III-1 to III-16.

In a particular embodiment, Q is a carbocycle or heterocycle selected from the group consisting of IIIa-IIIs:

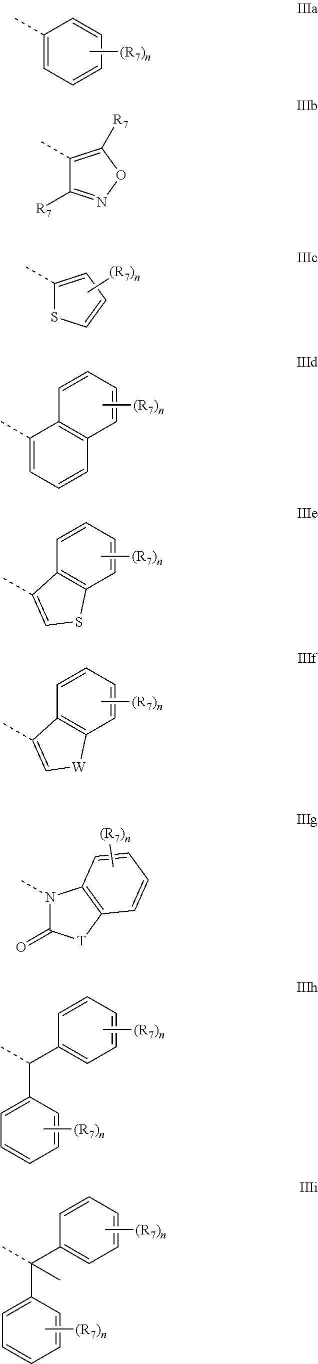

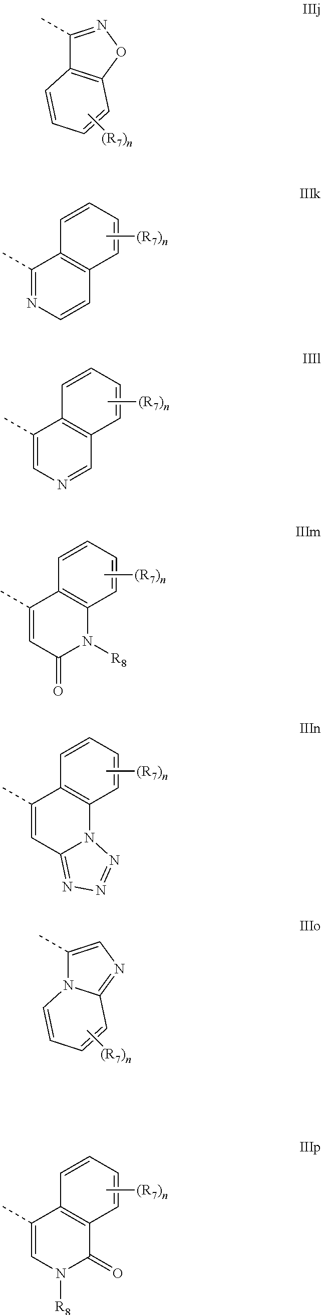

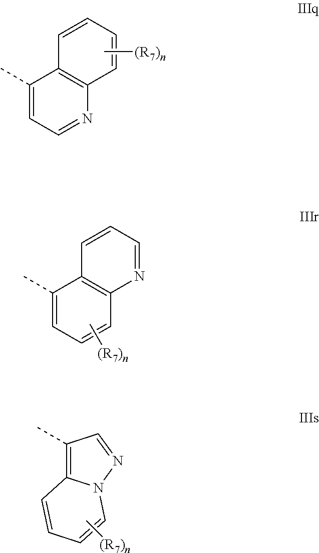

wherein n is 1-4, for example 1-3, for example 1-2, for example 1; T is O, S, NR₈ or CR₇R₇; W is O, NR₈ or CR₇R₇; and R₇ and R₈ are as defined herein. In a particular embodiment Q is any one of IIIa-IIIi wherein R₈ is H and R₇ is selected from the group consisting of H, F, Cl, Me, methoxy, hydroxyethoxy, methoxyethoxy, acetoxyethoxy, methylsulfonyl methylsulfonylmethyl, phenyl and morpholin-4-yl. In another particular embodiment Q is IIId. In a particular embodiment Q is IIId which is substituted at the 4-position with R₇. In another particular embodiment Q is IIId which is substituted at the 5-position with R₇. In a particular embodiment Q is F, Me, iPr, phenyl, phenyl substituted as follows: 2-Cl, 3-Cl, 4-Cl, 2-F, 3-F or 4-F substituted, benzyl, pyrid-3-yl or pyrid-4-yl. In an embodiment, one Q is a group of the general formula IIIa to IIIs.

R₁ is H, OH or alkyl; or R₁ and R₂ together form a 5-8 member heterocycle. In a particular embodiment, R₁ is H. In a particular embodiment, R₁ and R₂ together form a 6-member ring. In a particular embodiment, R₁ and R₂ together form a 7-member ring. In another particular embodiment, R₁ and R₂ together form an 8-member ring. In another particular embodiment, R₁ and R₂ together form a 7-member ring while Y is S. In another particular embodiment, R₁ is H, while Y is CH₂. In another particular embodiment, R₁ is H, while Y is S. In another particular embodiment, R₁ is H, while Y is O.

R₂ is alkyl, a carbocycle, carbocyclylalkyl, a heterocycle or heterocyclylalkyl each optionally substituted with halogen, hydroxyl, oxo, thione, mercapto, carboxyl, alkyl, haloalkyl, acyl, alkoxy, alkylthio, sulfonyl, amino and nitro, wherein said alkyl, acyl, alkoxy, alkylthio and sulfonyl are optionally substituted with hydroxy, mercapto, halogen, amino, alkoxy, hydroxyalkoxy and alkoxyalkoxy. In an embodiment, R₂ is alkyl, a carbocycle, carbocyclylalkyl, a heterocycle or heterocyclylalkyl each optionally substituted with halogen, hydroxyl, oxo, thione, mercapto, carboxyl, alkyl, haloalkyl, alkoxy, alkylthio, sulfonyl, amino and nitro. In a particular embodiment R₂ is alkyl, a carbocycle, carbocyclylalkyl, a heterocycle or heterocyclylalkyl each optionally substituted with halogen, hydroxyl, oxo, mercapto, thione, carboxyl, alkyl, haloalkyl, alkoxy, acyl, alkylthio, acyl, hydroxyacyl, methoxyacyl, sulfonyl, amino and nitro. In an embodiment R₂ is alkyl, a carbocycle, carbocyclylalkyl, a heterocycle or heterocyclylalkyl each optionally substituted with halogen, hydroxyl, mercapto, carboxyl, alkyl, alkoxy, acyl, amino and nitro. In a particular embodiment R₂ is alkyl, cycloalkyl, cycloalkylalkyl, aryl, aralkyl, a heterocycle or heterocyclylalkyl. In a particular embodiment R₂ is alkyl, cycloalkyl or a heterocycle. In a particular embodiment R₂ is selected from the group consisting of t-butyl, isopropyl, cyclohexyl, tetrahydropyran-4-yl, N-methylsulfonylpiperidin-4-yl, tetrahydrothiopyran-4-yl, tetrahydrothiopyran-4-yl (in which the S is in oxidized form SO or SO₂), cyclohexan-4-one, 4-hydroxycyclohexane, 4-hydroxy-4-methylcyclohexane, 1-methyl-tetrahydropyran-4-yl, 2-hydroxyprop-2-yl, but-2-yl, thiophen-3-yl, piperidin-4-yl, N-acetylpiperidin-4-yl, N-hydroxyethylpiperidin-4-yl, N-(2-hydroxyacetyl)piperidin-4-yl, N-(2-methoxyacetyl)piperidin-4-yl, pyridin-3-yl, phenyl, tetrahydrofuran-2-yl-carbonyl, methoxyethanone, 2-methoxyethoxyethanone and 1-hydroxyeth-1-yl. In an embodiment of the invention R₂ is t-butyl, isopropyl, cyclohexyl, cyclopentyl, phenyl or tetrahydropyran-4-yl. In a particular embodiment, R₂ is phenyl. In a particular embodiment, R₂ is cyclohexyl. In another embodiment R₂ is tetrahydropyran-4-yl. In another particular embodiment, R₂ is isopropyl (i.e. the valine amino acid side chain). In another particular embodiment, R₂ is t-butyl. In a particular embodiment R₂ is oriented such that the amino acid, or amino acid analogue, which it comprises is in the L-configuration.

R₃ is H or alkyl optionally substituted with halogen or hydroxyl; or R₃ and R₄ together form a 3-6 heterocycle. In an embodiment R₃ is H or alkyl; or R₃ and R₄ together form a 3-6 heterocycle. In an embodiment R₃ is H or methyl, ethyl, propyl or isopropyl. In a particularly particular embodiment R₃ is H or methyl. In another particular embodiment R₃ is methyl. In another particular embodiment R₃ is fluoromethyl. In another particular embodiment, R₃ is ethyl. In another particular embodiment R₃ is hydroxyethyl. In a particular embodiment R₃ is fluoromethyl. In a particular embodiment R₃ is hydroxyethyl. In another embodiment R₃ is oriented such that the amino acid, or amino acid analogue, which it comprises is in the L-configuration. In a particular embodiment R₃ and R₄ together with the atoms from which they depend form a 3-6 heterocycle. In a particular embodiment R₃ and R₄ together form an azetidine ring. In a particular embodiment R₃ and R₄ together form a pyrrolidine.

R₃′ is H, or R₃ and R₃′ together form a 3-6 carbocycle. In an embodiment, R₃′ is H. In another embodiment R₃ and R₃′ together form a 3-6 carbocycle, for example a cyclopropyl ring. In a particular embodiment R₃ and R₃′ are both methyl.

R₄ and R₄′ are independently H, hydroxyl, amino, alkyl, carbocycle, carbocycloalkyl, carbocycloalkyloxy, carbocycloalkyloxycarbonyl, heterocycle, heterocycloalkyl, heterocycloalkyloxy or heterocycloalkyloxycarbonyl; wherein each alkyl, carbocycloalkyl, carbocycloalkyloxy, carbocycloalkyloxycarbonyl, heterocycle, heterocycloalkyl, heterocycloalkyloxy and heterocycloalkyloxycarbonyl is optionally substituted with halogen, hydroxyl, mercapto, carboxyl, alkyl, alkoxy, amino, imino and nitro; or R₄ and R₄′ together form a heterocycle. In an embodiment R₄ and R₄′ are independently H, hydroxyl, amino, alkyl, aryl, aralkyl, cycloalkyl, cycloalkylalkyl, heteroaryl, or heteroarylalkyl wherein each alkyl, aryl, aralkyl, cycloalkyl, cycloalkylalkyl, heteroaryl and heteroarylalkyl is optionally substituted with halogen, hydroxyl, mercapto, carboxyl, alkyl, alkoxy, amino and nitro; or R₄ and R₄′ together form a heterocycle. In a particular embodiment R₄ and R₄′ together form a heterocycle, for example an azetidine ring, or a pyrrolidine ring. In a particular embodiment R₄ and R₄′ are both H. In another particular embodiment R₄ is methyl and R₄′ is H. In a particular embodiment one of R₄ and R₄′ is hydroxyl (OH) while the other is H. In another embodiment, one of R₄ and R₄′ is amino, such as NH₂, NHMe and NHEt, while the other is H. In a particular embodiment, R₄′ is H and R₄ is H, alkyl, aryl, aralkyl, cycloalkyl, cycloalkylalkyl, heteroaryl or heteroarylalkyl. In a particular embodiment R₄ is a group selected from the group consisting of:

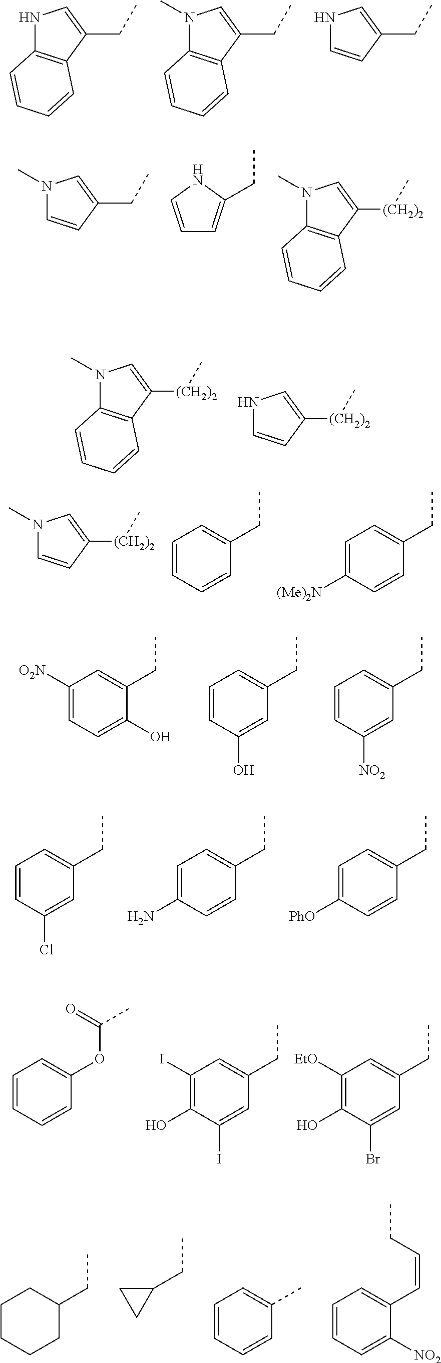

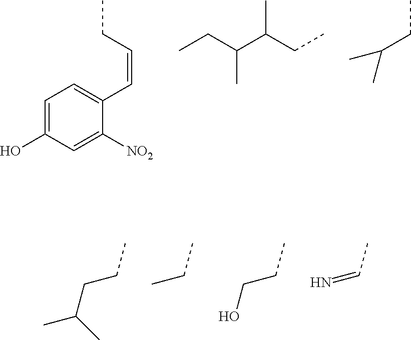

R₅ is H or alkyl. In a particular embodiment, R₅ is H or methyl. In a particular embodiment, R₅ is H. In another particular embodiment, R₅ is methyl.

R₆, and R₆′ are each independently H, alkyl, aryl or aralkyl. In a particular embodiment, R₆ is alkyl, for example methyl. In another particular embodiment R₆ is aryl, for example phenyl. In another particular embodiment R₆ is aralkyl, for example benzyl. In a particular embodiment R₆ and R₆′ are the same, for example both alkyl, e.g. both methyl. In another particular embodiment R₆ is methyl and R₆′ is H.

R₇ in each occurrence is independently H, cyano, hydroxyl, mercapto, halogen, nitro, carboxyl, amidino, guanidino, alkyl, a carbocycle, a heterocycle or —U-V; wherein U is —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)— and V is alkyl, a carbocycle or a heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)—; and an alkyl, carbocycle and heterocycle is optionally substituted with hydroxyl, alkoxy, acyl, halogen, mercapto, oxo, carboxyl, acyl, halo-substituted alkyl, amino, cyano, nitro, amidino, guanidino an optionally substituted carbocycle or an optionally substituted heterocycle. Substituents of the “optionally substituted carbocycle” and “optionally substituted heterocycle” are as defined herein. In a particular embodiment such carbocycle and heterocycle groups are substituted with hydroxyl, alkyl, alkoxy, acyl, halogen, mercapto, oxo, carboxyl, acyl, halo-substituted alkyl, amino, cyano, nitro, amidino and guanidino. In an embodiment R₇ is H, halogen, alkyl, haloalkyl, aryl, aralkyl, amino, arylamino, alkylamino, aralkylamino, alkoxy, alkoxyalkoxy, aryloxy or aralkyloxy.

R₈ is H, alkyl, a carbocycle or a heterocycle wherein one or more CH₂ or CH groups of said alkyl is optionally replaced with —O—, —S—, —S(O)—, S(O)₂, —N(R₈), or —C(O)—; and said alkyl, carbocycle and heterocycle is optionally substituted with hydroxyl, alkoxy, acyl, halogen, mercapto, oxo (═O), carboxyl, acyl, halo-substituted alkyl, amino, cyano nitro, amidino, guanidino an optionally substituted carbocycle or an optionally substituted heterocycle. Substituents of the “optionally substituted carbocycle” and “optionally substituted heterocycle” are as defined herein. In a particular embodiment such carbocycle and heterocycle groups are substituted with hydroxyl, alkyl, alkoxy, acyl, halogen, mercapto, oxo, carboxyl, acyl, halo-substituted alkyl, amino, cyano, nitro, amidino and guanidino. In a particular embodiment R₈ is H, alkyl, or acyl. In an embodiment R₈ is methyl. In another embodiment R₈ is acetyl. In a particular embodiment R₈ is H. In an embodiment R₇ is H, halogen, amino, hydroxyl, carboxyl, alkyl, haloalkyl or aralkyl. In a particular embodiment R₇ is halogen, for example Cl or F. In a particular embodiment R₇ is H. It is understood that substitutions defined for R₇ and R₈ as well as all other variable groups herein are subject to permissible valency.

R₉ is H, alkyl or acyl. In an embodiment R₉ is H. In an embodiment R₉ is alkyl. In an embodiment R₉ is acyl. In an embodiment R₉ is methyl. In an embodiment R₉ is acetyl.

m is 0 to 4. In an embodiment m is 0. In an embodiment m is 1. In an embodiment m is 2. In an embodiment m is 3. In an embodiment m is 4.

Compounds of the invention contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Diastereomeric compounds may be separated by chromatographic or crystallization methods. Similarly, enantiomeric mixtures may be separated using the same techniques or others known in the art. Each of the asymmetric carbon atoms may be in the R or configuration and both of these configurations are within the scope of the invention.

In particular embodiments, compounds of the invention have the general formula IIa-IId

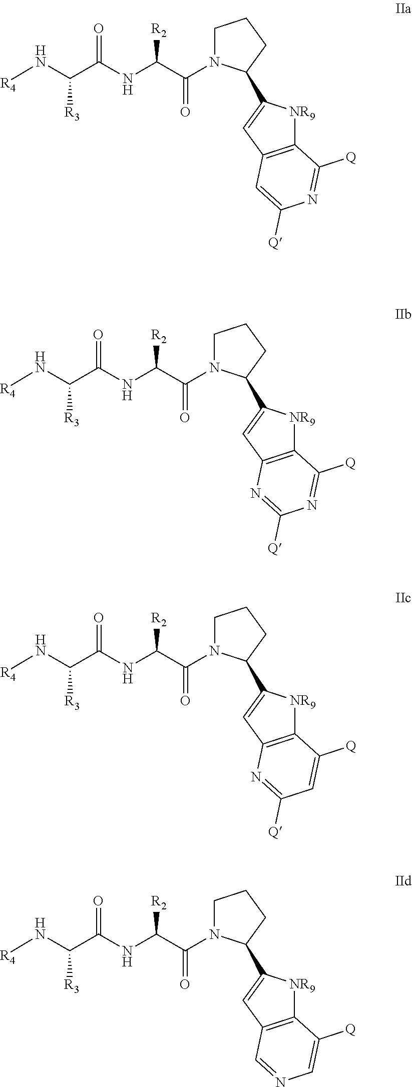

wherein Q, R₂, R₃, R₄, and R₉ are as described herein and Q′ is H, halogen, hydroxyl, carboxyl, amino, nitro, alkyl, a carbocycle or a heterocycle wherein said alkyl, carbocycle and heterocycle are optionally substituted with halogen, hydroxyl, carboxyl, amino, and nitro. In an embodiment Q′ is H. In an embodiment Q′ is alkyl, for example methyl, ethyl, propyl and isopropyl. In an embodiment Q′ is halogen. In an embodiment Q′ is F.

The invention also encompasses prodrugs of the compounds described above. Suitable prodrugs where applicable include known amino-protecting and carboxy-protecting groups which are released, for example hydrolyzed, to yield the parent compound under physiologic conditions. A particular class of prodrugs are compounds in which a nitrogen atom in an amino, amidino, aminoalkyleneamino, iminoalkyleneamino or guanidino group is substituted with a hydroxy (OH) group, an alkylcarbonyl (—CO—R) group, an alkoxycarbonyl (—CO—OR), an acyloxyalkyl-alkoxycarbonyl (—CO—O—R—O—CO—R) group where R is a monovalent or divalent group and as defined above or a group having the formula —C(O)—O—CP1P2-haloalkyl, where P1 and P2 are the same or different and are H, lower alkyl, lower alkoxy, cyano, halo lower alkyl or aryl. In a particular embodiment, the nitrogen atom is one of the nitrogen atoms of the amidino group of the compounds of the invention. These prodrug compounds are prepared reacting the compounds of the invention described above with an activated acyl compound to bond a nitrogen atom in the compound of the invention to the carbonyl of the activated acyl compound. Suitable activated carbonyl compounds contain a good leaving group bonded to the carbonyl carbon and include acyl halides, acyl amines, acyl pyridinium salts, acyl alkoxides, in particular acyl phenoxides such as p-nitrophenoxy acyl, dinitrophenoxy acyl, fluorophenoxy acyl, and difluorophenoxy acyl. The reactions are generally exothermic and are carried out in inert solvents at reduced temperatures such as −78 to about 50 C. The reactions are usually also carried out in the presence of an inorganic base such as potassium carbonate or sodium bicarbonate, or an organic base such as an amine, including pyridine, triethylamine, etc. One manner of preparing prodrugs is described in U.S. Ser. No. 08/843,369 filed Apr. 15, 1997 (corresponding to PCT publication WO9846576) the contents of which are incorporated herein by reference in their entirety.

Particular compounds of formula I include the following:

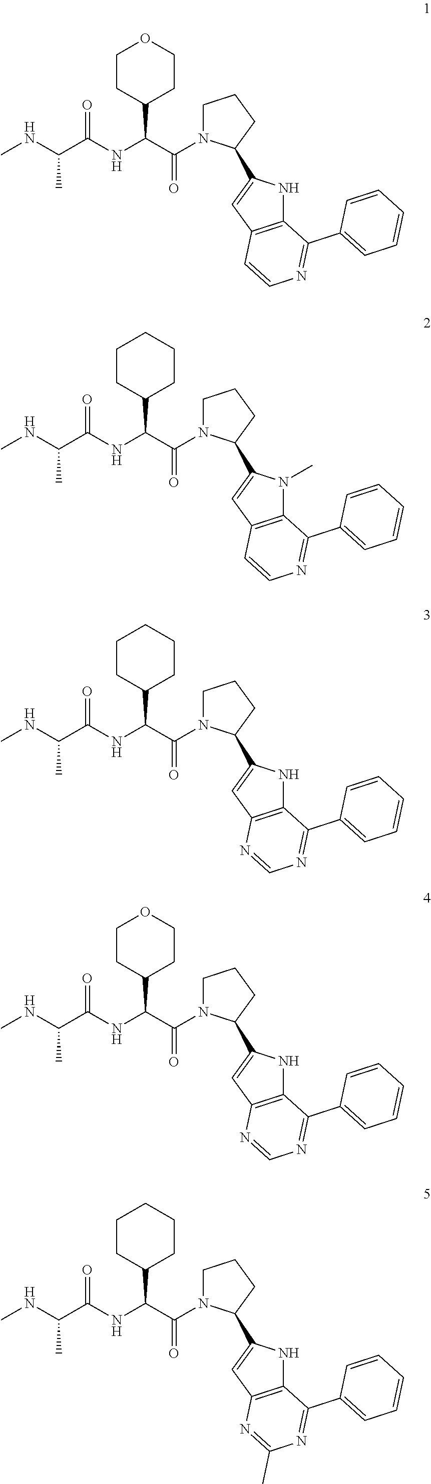

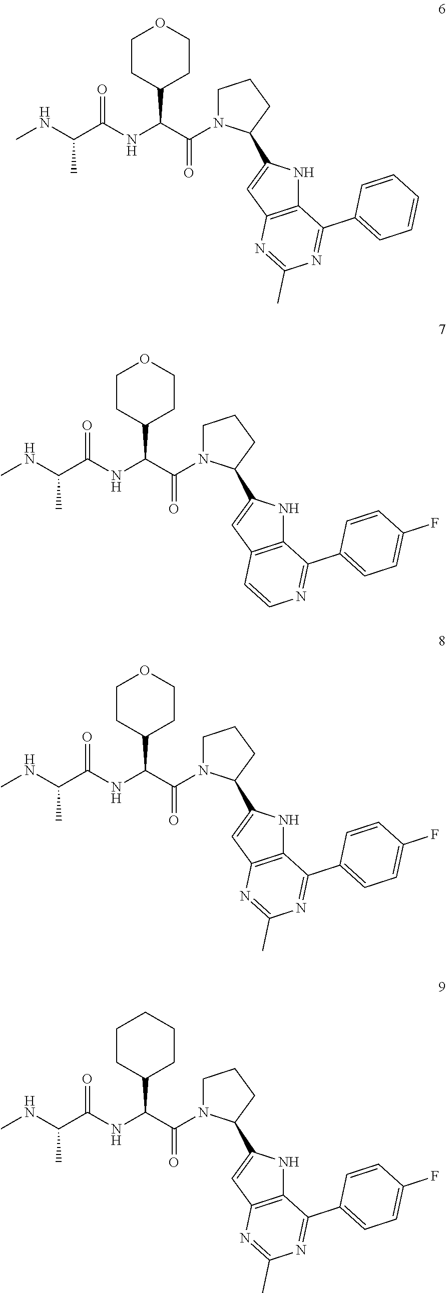

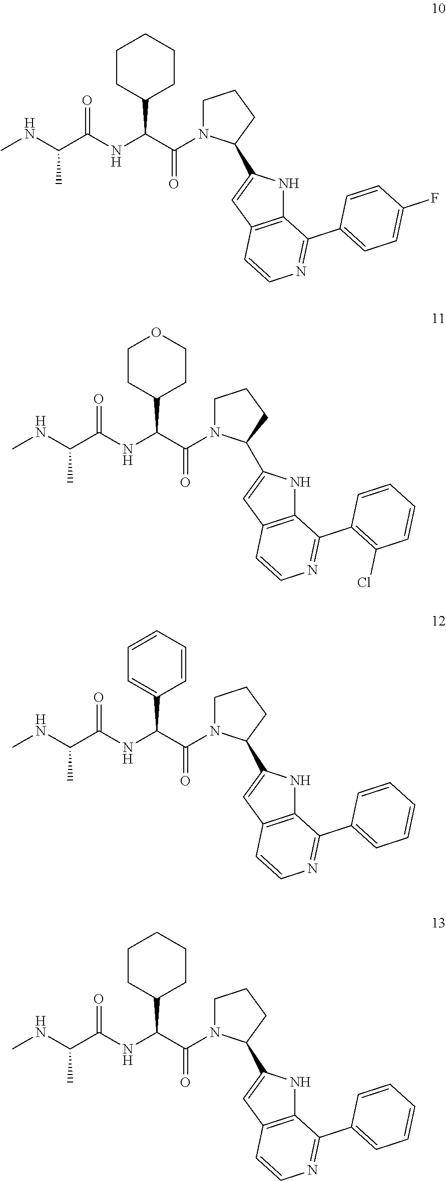

Compounds of the invention may exist in different resonance forms and that all such resonance forms are within the scope of the invention herein.

### Synthesis

Compounds of the invention are prepared using standard organic synthetic techniques from commercially available starting materials and reagents. It will be appreciated that synthetic procedures employed in the preparation of compounds of the invention will depend on the particular substituents present in a compound and that various protection and deprotection steps that are standard in organic synthesis may be required but may not be illustrated in the following general schemes. In a particular general synthetic scheme, compounds of the invention may be prepared by coupling amino acid residue analogues employing typical amide coupling procedures. In scheme 1, wherein X₁, X₂, Y, Z₁, Z₂, Z₃, Z₄, R₁, R₂, R₃, R₃′, R₄, R₄′, R₅, R₆, R₆′ and R₉ are as defined herein and Pr is a suitable protecting group, amine-protected amino acid residue analogues are coupled and deprotected sequentially to give the final compounds.

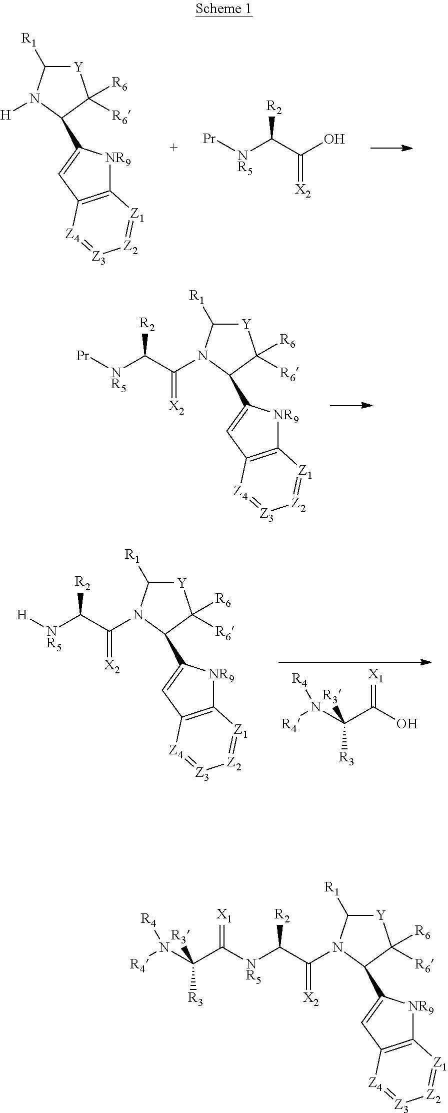

It will be appreciated that the amino acid analogs may be coupled in any order and may be prepared using solid phase support which is routine in the art. For example, Scheme 2 illustrates an alternative amino acid residue analogue coupling route.

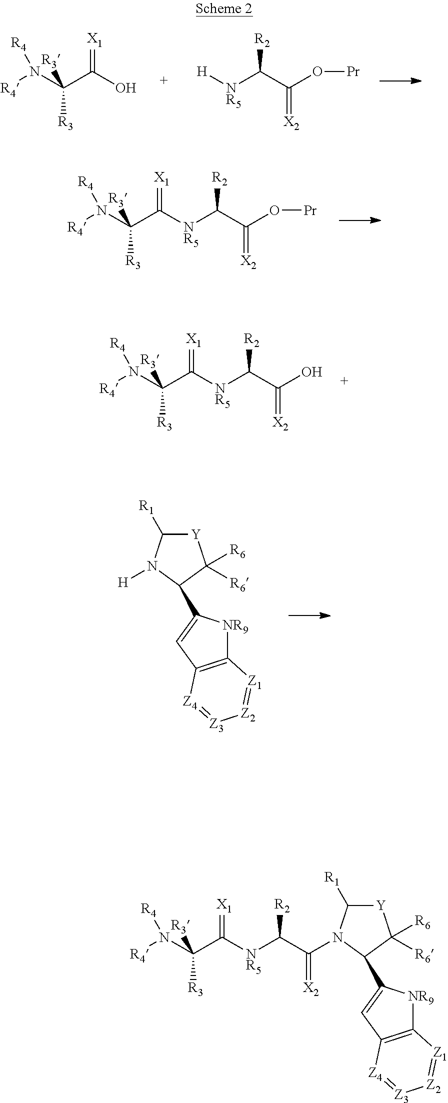

Azaindole intermediates may be prepared according to scheme 3 wherein Y, Z₁, Z₂, Z₃, Z₄, R₁, R₆, R_{6′} and R₉ are as defined herein.

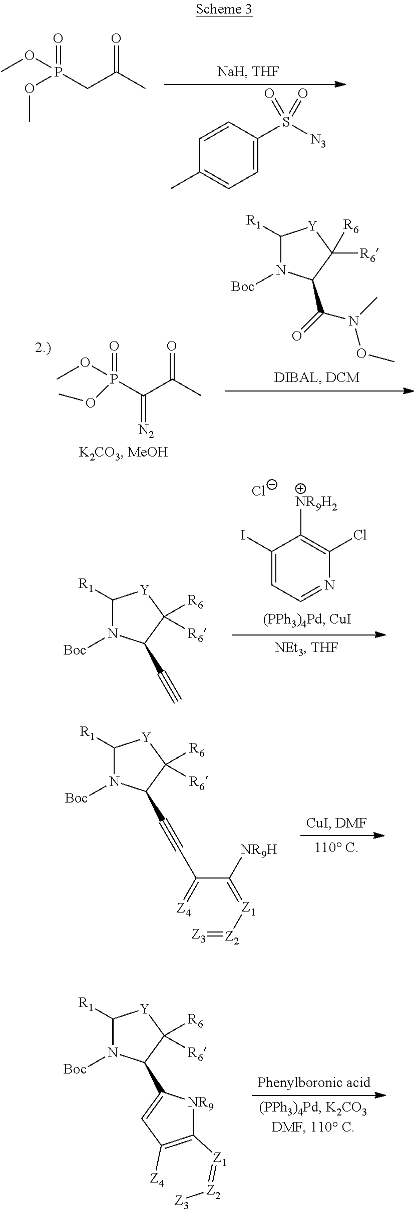

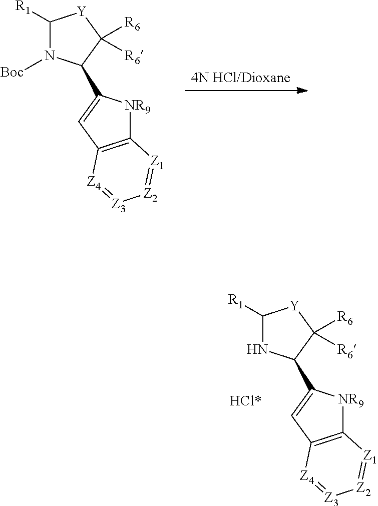

Compounds of the invention in which R₄ or R₄′ are other than H may be prepared according to standard organic chemistry techniques, for example by reductive amination in which a starting amino acid residue analog e.g. NH₂—CH(R₃)—C(O)—OH is reacted with a suitable aldehyde or ketone to give the desired R₄ and R₄′ substituents. See scheme 4. The resulting R₄/R₄′ substituted amino acid intermediate can then be conjugated to the next amino acid intermediate or the remainder of the compound using standard peptide coupling procedures.

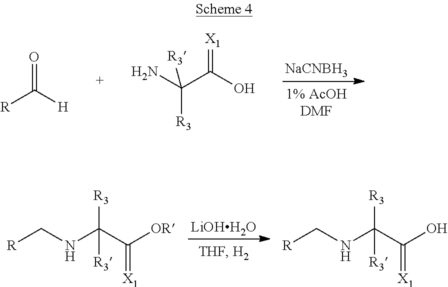

In a particular embodiment, alanine is reacted with 1-methylindole-2-carboxaldehyde and reduced with sodium cyanoborohydride dissolved in 1% HOAc/DMF to give the N-substituted alanine residue which may be used in preparing compounds of the invention. See scheme 5.

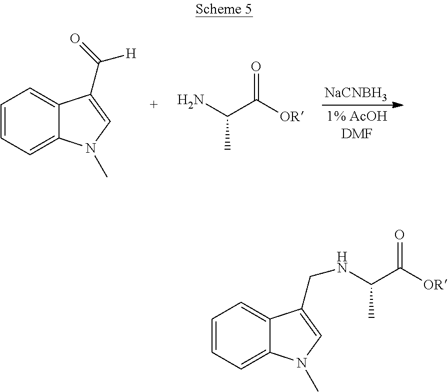

Alternatively, the reductive amination procedure to introduce R₄/R₄′ substituents is the final step in the preparation of the compound.

When compounds of the invention incorporate R₄ or R₄′ substituents other than H, they may also be prepared by substitution of a suitable acid intermediate which incorporates a leaving group with a desired amine For example Br—CH(R₃)—C(O)—OH is substituted with an amine R₄—NH₂ or R₄—NH—R₄′ according to scheme 6.

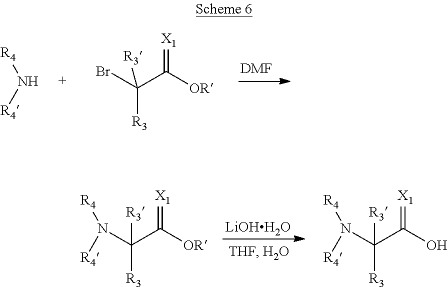

Alternatively, the substitution reaction introducing R₄ or R₄′ substituents may be performed as a final step in the preparation of the compound as illustrated in scheme 7.

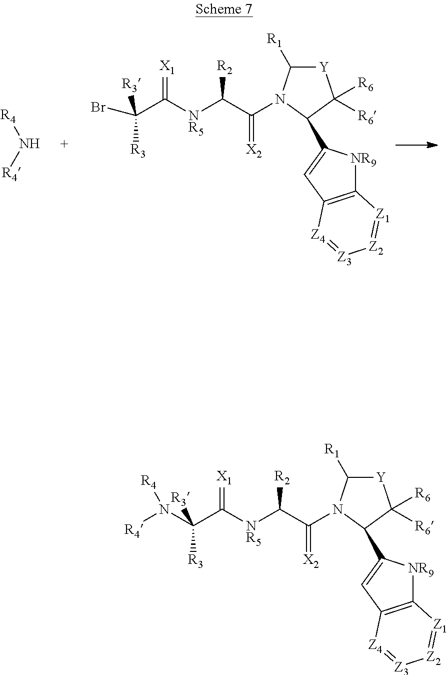

In a particular embodiment, the following amines used in schemes 6 and 7:

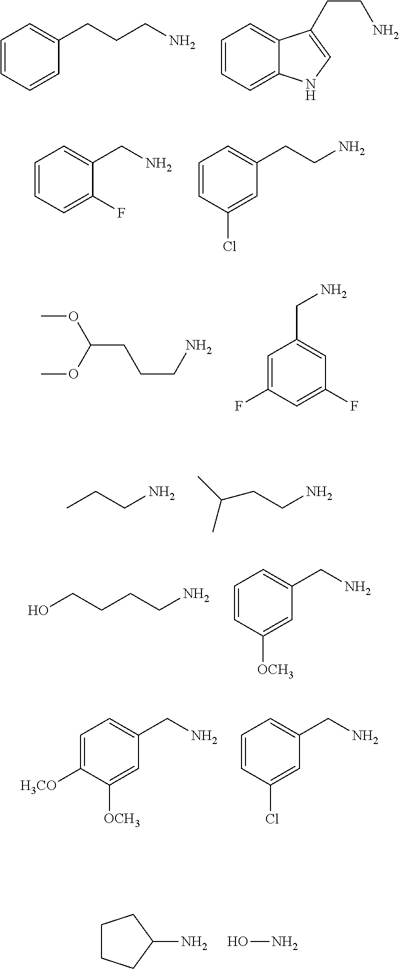

Compounds of the invention in which either X₁ or X₂ is sulfur, i.e. the compound incorporates a thioamide, may be prepared according to established organic chemistry techniques. For example, compounds in which X₂ is sulfur can be prepared according to scheme 8 starting from an Fmoc protected amino acid residue analog NH₂—CH(R₂)—COOH which is dissolved in THF and cooled to −25° C., with addition of DIPEA followed by addition of isobutylchloroformate. After 10 minutes, the diamine, 4-nitrobenzene-1,2-diamine, is added and the reaction mixture is continuously stirred at −25° C. for 2 hours, then at room temperature overnight. THF is vacuumed off and the mixture is then subjected to flash chromatography using 50% EtOAc/Hexane to yield the product. The Fmoc-alanine derivative, phosphorus pentasulfide and sodium carbonate are mixed in THF and stirred overnight. The solution is concentrated and direct chromatography using 80% EtOAc/Hexane yields the activated thioalanine. The activated thioalanine and sodium nitrite are then mixed in acetic acid and diluted with H₂O. The resulting precipitant is filtered and dried to yield the product. The thioalanine is coupled to an A ring substitued proline amino acid residue analog by dissolving both in DMF. The thioamide product may then be deprotected with 20% PIP/DMA for 15 minutes and used to conjugate to the R₄/R₄′—N—C(R₃)(R₃′)—COOH.

### Indications

The compounds of the invention inhibit the binding of IAP proteins to caspases, in particular XIAP binding interaction with caspases 3 and 7. The compounds also inhibit the binding of ML-IAP to Smac protein. Accordingly, the compounds of the invention are useful for inducing apoptosis in cells or sensitizing cells to apoptotic signals, in particular cancer cells. Compounds of the invention are useful for inducing apoptosis in cells that overexpress IAP proteins. Alternatively, compounds of the invention are useful for inducing apoptosis in cells in which the mitochondrial apoptotic pathway is disrupted such that release of Smac from mitochondia is inhibited, for example by up regulation of Bcl-2 or down regulation of Bax/Bak. More broadly, the compounds can be used for the treatment of all cancer types which fail to undergo apoptosis. Examples of such cancer types include neuroblastoma, intestine carcinoma such as rectum carcinoma, colon carcinoma, familiarly adenomatous polyposis carcinoma and hereditary non-polyposis colorectal cancer, esophageal carcinoma, labial carcinoma, larynx carcinoma, hypopharynx carcinoma, tong carcinoma, salivary gland carcinoma, gastric carcinoma, adenocarcinoma, medullary thyroidea carcinoma, papillary thyroidea carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors such as glioblastoma, astrocytoma, meningioma, medulloblastoma and peripheral neuroectodermal tumors, Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia lymphoma, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondro sarcoma, myosarcoma, liposarcoma, fibrosarcoma, Ewing sarcoma and plasmocytoma.

In an embodiment, compounds of the invention selectively bind XIAP relative to cIAP1 as measured in a binding assay such as a Time-Resolved Fluorescence Resonance Energy Transfer (TR-FRET assay or a Fluorescence Polarization assay as described herein. In a particular embodiment, compounds of the invention have >10-fold greater binding affinity for XIAP relative to cIAP1. In another particular embodiment, compounds of the invention have >100-fold greater binding affinity to XIAP. In a particular embodiment, compounds of the invention have >1000-fold greater binding affinity to XIAP.

Compounds of the invention are useful for sensitizing cells to apoptotic signals. Accordingly, the compounds may be administered prior to, concomitantly with, or following administration of radiation therapy or cytostatic or antineoplastic chemotherapy. Suitable cytostatic chemotherapy compounds include, but are not limited to (i) antimetabolites, such as cytarabine, fludarabine, 5-fluoro-2′-deoxyuiridine, gemcitabine, hydroxyurea or methotrexate; (ii) DNA-fragmenting agents, such as bleomycin, (iii) DNA-crosslinking agents, such as chlorambucil, cisplatin, cyclophosphamide or nitrogen mustard; (iv) intercalating agents such as adriamycin (doxorubicin) or mitoxantrone; (v) protein synthesis inhibitors, such as L-asparaginase, cycloheximide, puromycin or diphtheria toxin; (Vi) topoisomerase I poisons, such as camptothecin or topotecan; (vii) topoisomerase II poisons, such as etoposide (VP-16) or teniposide; (viii) microtubule-directed agents, such as colcemid, colchicine, paclitaxel, vinblastine or vincristine; (ix) kinase inhibitors such as flavopiridol, staurosporin, STI571 (CPG 57148B) or UCN-01 (7-hydroxystaurosporine); (x) miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; (xi) hormones such as glucocorticoids or fenretinide; (xii) hormone antagonists, such as tamoxifen, finasteride or LHRH antagonists. In a particular embodiment, compounds of the present invention are coadministered with a cytostatic compound selected from the group consisting of cisplatin, doxorubicin, taxol, taxotere and mitomycin C. In a particular embodiment, the cytostatic compound is doxorubicin.

Another class of active compounds which can be used in the present invention are those which are able to sensitize for or induce apoptosis by binding to death receptors (“death receptor agonists”). Such agonists of death receptors include death receptor ligands such as tumor necrosis factor a (TNF-α), tumor necrosis factor β (TNF-β, lymphotoxin-α), LT-β (lymphotoxin-β), TRAIL (Apo2L, DR4 ligand), CD95 (Fas, APO-1) ligand, TRAMP (DR3, Apo-3) ligand, DR6 ligand as well as fragments and derivatives of any of said ligands. In an embodiment, the death receptor ligand is TNF-α. In a particular embodiment, the death receptor ligand is Apo2L/TRAIL. Furthermore, death receptors agonists comprise agonistic antibodies to death receptors such as anti-CD95 antibody, anti-TRAIL-R1 (DR4) antibody, anti-TRAIL-R2 (DR5) antibody, anti-TRAIL-R3 antibody, anti-TRAIL-R4 antibody, anti-DR6 antibody, anti-TNF-R1 antibody and anti-TRAMP (DR3) antibody as well as fragments and derivatives of any of said antibodies.

For the purpose of sensitizing cells for apoptosis, the compounds of the present invention can be also used in combination with radiation therapy. The phrase “radiation therapy” refers to the use of electromagnetic or particulate radiation in the treatment of neoplasia. Radiation therapy is based on the principle that high-dose radiation delivered to a target area will result in the death of reproducing cells in both tumor and normal tissues. The radiation dosage regimen is generally defined in terms of radiation absorbed dose (rad), time and fractionation, and must be carefully defined by the oncologist. The amount of radiation a patient receives will depend on various consideration but the two most important considerations are the location of the tumor in relation to other critical structures or organs of the body, and the extent to which the tumor has spread. Examples of radiotherapeutic agents are provided in, but not limited to, radiation therapy and is known in the art (Hellman, Principles of Radiation Therapy, Cancer, in Principles I and Practice of Oncology, 24875 (Devita et al., 4th ed., vol 1, 1993). Recent advances in radiation therapy include three-dimensional conformal external beam radiation, intensity modulated radiation therapy (IMRT), stereotactic radiosurgery and brachytherapy (interstitial radiation therapy), the latter placing the source of radiation directly into the tumor as implanted “seeds”. These newer treatment modalities deliver greater doses of radiation to the tumor, which accounts for their increased effectiveness when compared to standard external beam radiation therapy. Ionizing radiation with beta-emitting radionuclides is considered the most useful for radiotherapeutic applications because of the moderate linear energy transfer (LET) of the ionizing particle (electron) and its intermediate range (typically several millimeters in tissue). Gamma rays deliver dosage at lower levels over much greater distances. Alpha particles represent the other extreme, they deliver very high LET dosage, but have an extremely limited range and must, therefore, be in intimate contact with the cells of the tissue to be treated. In addition, alpha emitters are generally heavy metals, which limits the possible chemistry and presents undue hazards from leakage of radionuclide from the area to be treated. Depending on the tumor to be treated all kinds of emitters are conceivable within the scope of the present invention.

Furthermore, the present invention encompasses types of non-ionizing radiation like e.g. ultraviolet (UV) radiation, high energy visible light, microwave radiation (hyperthermia therapy), infrared (IR) radiation and lasers. In a particular embodiment of the present invention UV radiation is applied.

The invention also includes pharmaceutical compositions or medicaments containing the compounds of the invention and a therapeutically inert carrier, diluent or excipient, as well as methods of using the compounds of the invention to prepare such compositions and medicaments. Typically, the compounds of formula I used in the methods of the invention are formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but may range anywhere from about 3 to about 8. Formulation in an acetate buffer at pH 5 is a suitable embodiment. In an embodiment, the inhibitory compound for use herein is sterile. The compound ordinarily will be stored as a solid composition, although lyophilized formulations or aqueous solutions are acceptable.

The composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to inhibit IAP interaction with caspases, induce apoptosis or sensitize a malignant cell to an apoptotic signal. Such amount is may be below the amount that is toxic to normal cells, or the mammal as a whole.

Generally, the initial pharmaceutically effective amount of the compound of the invention administered parenterally per dose will be in the range of about 0.01-100 mg/kg, for example about 0.1 to 20 mg/kg of patient body weight per day, with the typical initial range of compound used being 0.3 to 15 mg/kg/day. Oral unit dosage forms, such as tablets and capsules, may contain from about 25 to about 1000 mg of the compound of the invention.

The compound of the invention may be administered by any suitable means, including oral, topical, transdermal, parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (PVP) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g. a salt such sodium chloride, if desired. The solution is typically filtered, e.g. using a 0.2 micron filter, to remove impurities and contaminants.

### EXAMPLES

The invention will be more fully understood by reference to the following examples. They should not, however, be construed as limiting the scope of the invention. Reagents and solvents were obtained from commercial sources and used as received. Unjless otherwise noted, chromatographic purifications were performed using pre-packed silica gel columns on a CombiFlash Companion system by Teledyne-Isco, Inc. Lincoln, Nebr. The identity and purity of compounds were checked by LCMS and ¹H NMR analysis.

Abbreviations used herein are as follows:
AcOH: acetic acid;
ACN: acetonitrile;
Chg: cyclohexylglycine;
DCM: dichloromethane
DIPEA: diisopropylethylamine;
DMAP: 4-dimethylaminopyridine;
DME: 1,2-dimethoxyethane;
DMF: dimethylformamide;
DMSO: dimethylsulfoxide
EDC: 1-ethyl-3-(3-dimethylaminopropyl)carbodiimide;
EEDQ: 2-ethoxy-1-ethoxycarbonyl-1,2-dihydroquinoline;
EtOAc: ethylacetate
EtOH: ethanol;
LCMS: liquid chromatography mass spectrometry;
HATU: O-(7-Azobenzotriazol-1-yl)-1,1,3,3-tetramethyluronium hexafluorophosphate;
HOBt: N-Hydroxybenzotriazole
HBTU: 2-(1H-Benzotriazol-1-yl)-1,1,3,3-Tetramethyl-uronium Hexafluorophosphate;
HPLC: high performance liquid chromatography;
MeOH: methanol;
NBS: N-bromosuccinamide;
TASF: tris(dimethylamino)sulfonium difluorotrimethylsilicate;
TEA: triethylamine;
TFA: trifluoroacetic acid;
THF: tetrahydrofuran;

### Example 1

### 2-[tert-Butoxycarbonyl-(1H-pyrrol-2-ylmethyl)-amino]-propionic acid

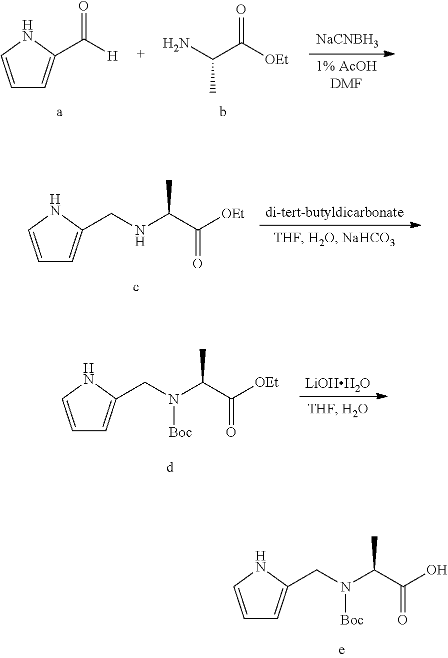

Alanine ethyl ester b (5 g, 32.5 mmol), pyrrole-2-carboxaldehyde a (3.1 g, 32.5 mmol), sodium cyanoborohydride (2.04 g, 32.5 mmol) and AcOH (1%) were mixed in DMF and stirred overnight. The reaction was quenched with H₂O, and DMF was evaporated. The mixture was diluted with EtOAc, washed by 0.1N NaOH, dried and concentrated to yield product c 2.5 g. The resulting ester c (2.5 g, 12.8 mmol), di-tert-butyldicarbonate (3.06 g, 14 mmol) were mixed in THF, H₂O with NaHCO₃ and stirred overnight. THF was evaporated, and the mixture was diluted with EtOAc, washed by 1N NaOH, sat. NH₄Cl and brine. After dried, the mixture was concentrated to yield the Boc-protected ester d 3.3 g. The Boc-protected ester d (1.67 g, 5.6 mol), lithium hydroxide mono hydrate (284 mg, 6.77 mmol) were mixed in THF and H₂O at 0° C. THF was vacuumed off, and the solution was acidified by dilute H₂SO₄, extracted by EtOAc twice. Organic layers were combined, dried and evaporated giving product 2-[tert-butoxycarbonyl-(1H-pyrrol-2-ylmethyl)-amino]-propionic acid e.

### Example 2

### Tetrahydropyranylglycine

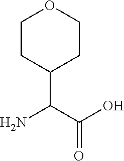

Tetrahydropyranylglycine was purchased from NovaBiochem, or synthesized according to the literature: Ghosh, A. K.; Thompson, W. J.; holloway, M. K.; McKee, S. P.; Duong, T. T.; Lee, H. Y.; Munson, P. M.; Smith, A. M.; Wai, J. M; Darke, P. L.; Zugay, J. A.; Emini, E. A.; Schleife, W. A.; Huff, J. R.; Anderson, P. S. J. Med. Chem., 1993, 36, 2300-2310.

### Example 3

### Piperidinylglycine

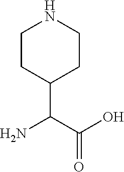

Piperidinylglycine was synthesized according to the procedures described by Shieh et al. (Tetrahedron: Asymmetry, 2001, 12, 2421-2425.

### Example 4

### 4,4-difluorocyclohexylglycine

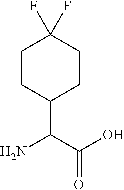

4,4-difluorocyclohexylglycine was made according to the procedures described in patent application US 20030216325.

### Example 5

### Boc (S)-2-amino-2-(4-hydroxycyclohexyl)acetic acid

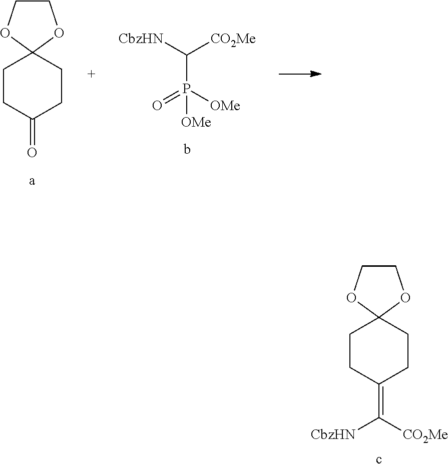

Following the procedure described by Sheih et al. (Tetrahedron: Asymmetry, 2001, 12, 2421-2425), a solution of ketone a (8.4 g) and EtOAc (30 mL) was added to a solution of N-Cbz-phosphonoglycine methyl ester b, TMG (4.5 mL) and EtOAc (30 mL). The solution was maintained at rt for 48 h, then washed with 1N HCl (3×50 mL), brine (1×50 mL) dried (Na₂SO₄), filtered, and concentrated. The residue was adsorbed onto Celite, and purified by chromatography, then further purified by re-crystallization from EtOAc/hexanes to afford 5.2 g of product c.

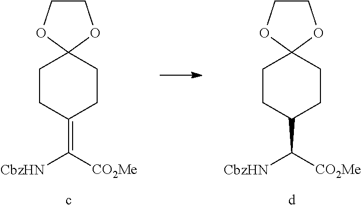

Following the procedure described by Sheih, (Tetrahedron: Asymmetry, 2001, 12, 2421-2425), a solution of eneamide c (5.0 g), (S,S)-Me-BPE-Rh(I) (1.5 g, Strem Chemicals, Newburyport, Mass.), and MeOH (100 mL) was shaken vigorously under 70 psi of H₂ for 48 h. The solvent was removed under reduced pressure. The residue was taken up in EtOAc, and filtered through SiO₂ with more EtOAc. The solvent was removed under reduced pressure to afford 4.0 g of product d as a colorless solid.

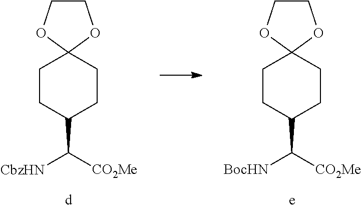

A mixture of Cbz-carbamate d, (4.0 g) Boc₂O, (2.9 g), 20% Pd(OH)₂.C (1.0 g) and MeOH (30 mL) was maintained under an atmosphere of H₂ for 6 h. The mixture was filtered through Celite with MeOH. The solvent was removed under reduced pressure to afford 4.5 g of residue e, which was taken on directly.

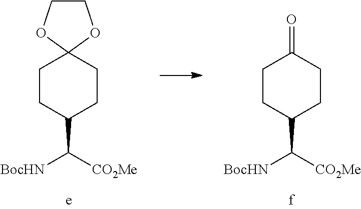

The residue e from above was dissolved in H₂O (10 mL), AcOH (30 mL), THF (5 mL), and dichloroacetic acid (3 mL) and maintained at rt overnight. Water (5 mL) was added and the solution and maintained until hydrolysis was complete, as monitored by HPLC-MS. Solid Na₂CO₃ was added cautiously until gas evolution ceased, the mixture was diluted with aq NaHCO₃, and extracted with 10% EtOAc/DCM. The combined organic phases were washed once with brine, dried (Na₂SO₄), filtered, and concentrated. The residue was purified by chromatography to afford 2.9 g of product f.

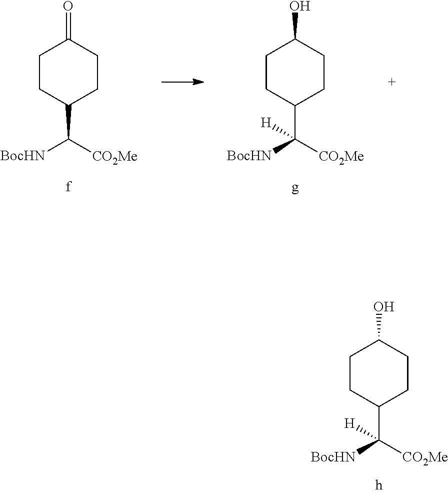

A mixture of ketone f (1.5 g) MeOH (50 ml) was treated with NaBH4 (290 mg) at 0° C. for 20 min. The mixture was acidified to ˜pH1 with 10% aq citric acid and the MeOH was removed under reduced pressure. The residue was diluted with water and extracted with 20% EtOAc/DCM. The combined organic phases were washed once with brine, dried (Na₂SO₄), filtered, and concentrated. The residue was purified by chromatography to afford 1.17 g of product g and 0.23 g of product h.

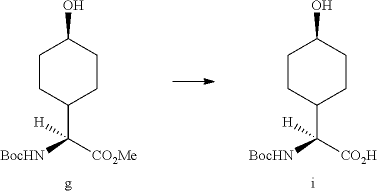

A mixture of ester g (1.17 g) LiOH.H₂O (160 mg), THF (3 mL) and water (4.5 mL) was stirred vigorously at rt overnight. The mixture was diluted with brine and exaustivly extracted with EtOAc. The combined organic phases were washed once with brine, dried (Na₂SO₄), filtered, and concentrated to afford acid i (525 mg).

### Example 6

### N-Boc-N-cyclopropylmethyl-L-alanine

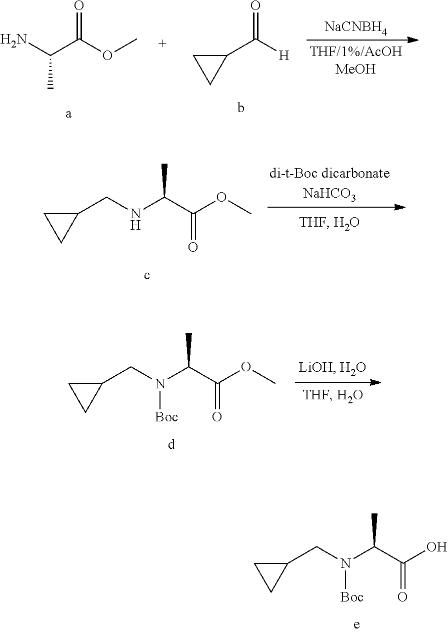

L-alanine methyl ester hydrochloride a (5 g, 35.8 mmol) and cyclopropanecarboxaldehyde b (2.67 ml, 35.8 mmol) were suspended in 50 ml THF w/1% AcOH. Addition of 5 ml of CH₃OH made the cloudy solution turned to clear. NaCNBH₄ (2.25 g, 35.8 mmol) was added and the reaction mixture stirred overnight. The reaction was quenched by addition of 1N aq. NaOH, extracted by EtOAc twice, organic layers were dried over Na₂SO₄ and concentrated to dryness. The crude material was purified by chromatography using 30% EtOAc/hexane (stained by ninhydrin) to obtain the compound c (1 g, 18%). The compound c (1 g, 6.37 mmol) and di-t-bocdicarbonate (2.1 g, 9.55 mmol) were diluted in THF (20 ml) and H₂O (20 ml), NaHCO₃ (1.3 g, 15.9 mmol) was added. The reaction mixture stirred overnight for completion. THF was removed under reduced pressure, and the aqueous layer was extracted by EtOAc 3 times. Combined organic layers were washed by 1N NaOH, sat, NH₄Cl followed by brine, the concentrated to dryness. The Boc-protected compound d (1.39 g, 5.40 mmol) was stirred with LiOH.H₂O (1.14 g, 27 mmol) in THF (20 ml) and H₂O (20 ml) overnight at room temperature. THF was stripped off, and the aqueous layer was adjusted to pH=4 by adding 10% citric acid, then extracted by EtOAc 3 times. Combined organic layers were washed by brine and concentrated. The crude was purified by reverse phase C-18 column eluted by 0%-50% acetonitrile/H₂O to give pure compound e as a white solid (794 mg).

### Example 7

### N-Boc-N-methyl-L-alanine-L-cyclohexylglycine

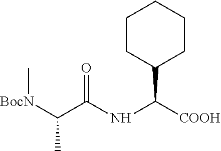

A solution of Fmoc-L-cyclohexylglycine (3.6 g, 9.6 mmol) dissolved in DCM (50 mL) and DIPEA (5.6 mL, 32 mmol) was added to 2-chlorotrityl chloride resin (5 g, 8 mmol) and gently agitated for 3 hours at room temperature. The resin was washed with DCM 4 times, DCM/MeOH/DIPEA (17:2:1) 3 times, DCM 3 times, and 2 times dimethylacetamide (DMA). The Fmoc group was removed by treating the resin with 20% piperidine/DMA (50 mL) for 15 minutes. The resin was washed with DMA 6 times. A solution of Boc-N-methylalanine (3.3 g, 16 mmol), HBTU (6.1 g, 16 mmol), and DIPEA (5.6 mL, 32 mmol) and DMA/DCM (1:1, 50 mL) was added to the resin and gently agitated for 2 hours at room temperature. The resin was washed with DMA 5 times, DCM 2 times, and dried under reduced pressure. The dipeptide was cleaved from the resin by gentle agitation with HOAc/TFE/DCM (1:1:3, 100 mL) for 2 hours at room temperature. The resin was removed by filtration and the solution concentrated. Residual AcOH was removed by azeotroping with hexanes (15 times volume). The solid residue was purified by reverse-phase HPLC (C₁₈, MeCN—H₂O, 0.1% TFA) and the solvents removed by lyophylization to provide 1.2 g (43%) of dipeptide N-Boc-N-methyl-L-alanine-L-cyclohexylglycine as a white powder.

### Example 8

### N-Boc-N-methyl-L-alanine-L-dehydropyranylglycine

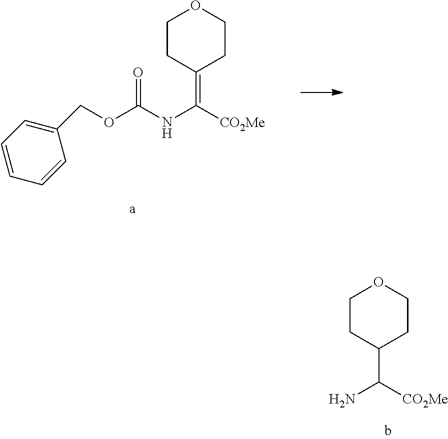

A mixture of N-Cbz-dehydropyranylglycine methyl ester a (Burk, M. J.; Gross, M. F.; Martinez, J. P. J. Am. Chem. Soc. 1995, 117, 9375, and references therein) (5.2 g, 17 mmol), 5% Pd.C (500 mg), MeOH (75 mL) and THF (25 mL) was maintained under an atmosphere of H₂ for 24 h. The mixture was filtered through Celite and the Celite washed with MeOH, and concentrated under reduced pressure to afford a quantitative yield of amine b as a colorless oil, which was carried on directly.

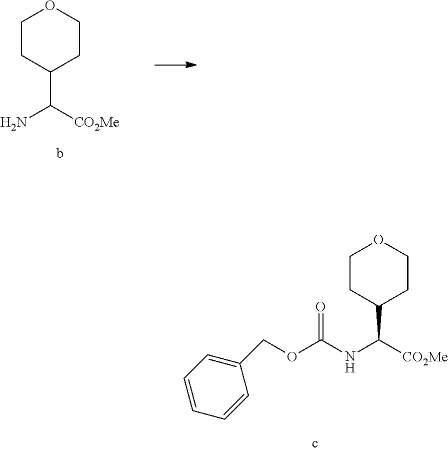

The amine b prepared above was combined with CH₂Cl₂ (40 mL), saturated aqueous NaHCO₃ (40 mL) and cooled to 0° C. Benzyloxy carbonyl chloride (3.0 mL) was then added dropwise and the mixture stirred vigorously overnight. The phases were separated and the aqueous phase extracted with CH₂Cl₂ (3×20 mL). The combined organic phases were washed with brine (1×50 mL), dried (Na₂SO₄), filtered, adsorbed onto Celite and chromatographed (ISCO, 120 g silica column, gradient elution 5-55% EtOAc-hexanes) to afford 4.15 g (80%) of racemic Cbz-pyranylglycine methyl ester. The enantiomers were separated on a Chiracel OD column eluting with 10% EtOH-hexanes. The desired S-enantiomer c elutes first under these conditions.

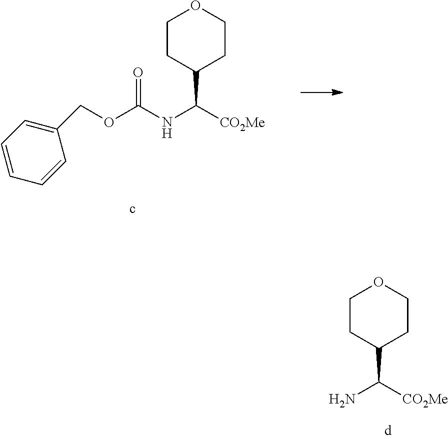

A mixture of (S)—N-Cbz-pyranyl glycine c methyl ester (2.4 g, 7.82 mmol) 10% Pd.C (700 mg), MeOH (80 mL) was maintained under 1 atmosphere of H₂ for 24 h. The mixture was filtered through Celite with MeOH, and concentrated under reduced pressure to afford 1.35 g (100%) of amine d as a colorless oil. Alternatively, pyranyl glycine can be synthesized in enantiopure form following the procedure of Ghosh (Ghosh, A. K.; Thompson, W. J.; Holloway, M. K.; McKee, S. P.; Duong, T. T.; Lee, H. Y.; Munson, P. M.; Smith, A. M.; Wai, J. M.; Darke, P. L.; Zugay, J. A.; Imini, E. A.; Schleif, W. A.; Huff, J. R.; Anderson, P. S. J. Med. Chem., 1993, 36, 2300).

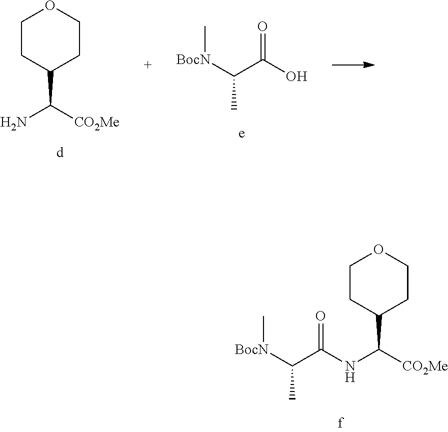

A mixture of amine d (1.35 g, 7.8 mmol), N-Boc-N-methyl alanine e (1.74 g, 8.6 mmol), EDC (1.65 g 8.8 mmol) and MeCN (50 mL) was maintained at rt overnight. The MeCN was removed under reduced pressure, and the residue diluted with EtOAc, washed with 0.5 N HCl (3×10 mL), 0.5 N NaOH (3×10 mL), dried (MgSO₄), filtered, and concentrated to provide 2.1 g (75%) of protected dipeptide f, as a clear oil.

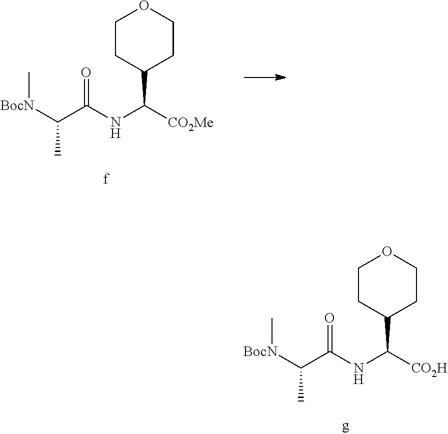

To a 0° C. solution of ester f (2.10 g, 5.86 mmol) and THF (50 mL) were added LiOH.H₂O (1.23 g, 29.3 mmol) and water (2 mL). The mixture was maintained at 0° C. for 2 h, then the cooling bath was removed and the mixture was stirred overnight. Most of the THF was then removed under reduced pressure and the residue was diluted with CH₂Cl₂, washed with 0.5 N HCl, dried (MgSO₄), filtered, and concentrated to provide 1.53 g (78%) of dipeptide N-Boc-N-methyl-L-alanine-L-dehydropyranylglycine g, as a colorless solid.

### Example 9

### N-Boc-Protected Cyclic Sulfonyl Amino Acid

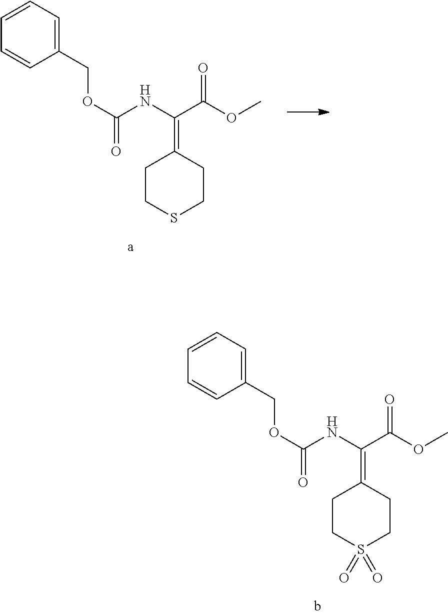

Sulfide a (810 mg, 2.5 mmol), synthesized according to the general procedure of Shieh [Shieh, W-C.; Xue, S.; Reel, N.; Wu, R.; Fitt, J.; Repic, O. Tetrahedron: Asymmetry, 2001, 12, 2421-2425], was dissolved in methanol (25 mL). Oxone (4.5 g) was dissolved in deionized water (25 mL).

The methanol solution of substrate was cooled to −10° C., and the aqueous solution of oxone was added to the reaction slowly. The reaction was kept on ice and gradually allowed to warm to room temperature while stirring overnight. Deionized water was used to dilute the reaction to approximately 150 mL, then poured into 90% ethyl acetate-hexanes for extraction. The organic phase was dried (Na₂SO₄), adsorbed onto Celite and purified by chromatography ISCO CombiFlash 40 g column, 5-90% ethyl acetate-hexanes over 30 min to afford 804 mg (2.27 mmol, 91%) of the product sulfone b.

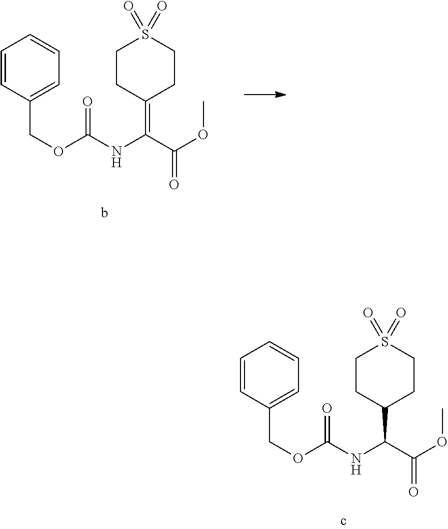

Following the general procedure of Burk [Burk, M. J.; Gross, M. F.; Martinez, J. P. J. Am. Chem. Soc. 1995, 117, 9375-9376.], alkene b (774 mg 2.19 mmol), dry methanol (40 mL), and [(S,S)-Me-BPE-Rh(CON)]⁺OTf⁻ (500 mg, 0.8 mmol) were mixed in a Parr shaker flask purged with nitrogen. The Parr flask was evacuated and subsequently charged to 60 psi with hydrogen gas and shaken vigorously overnight. Methanol was removed under reduced pressure, and crude product was filtered through a small plug of silica gel using ethyl acetate. Evaporation of the solvent yielded 730 mg (2.0 mmol, 94%) of product c with >98% yield.

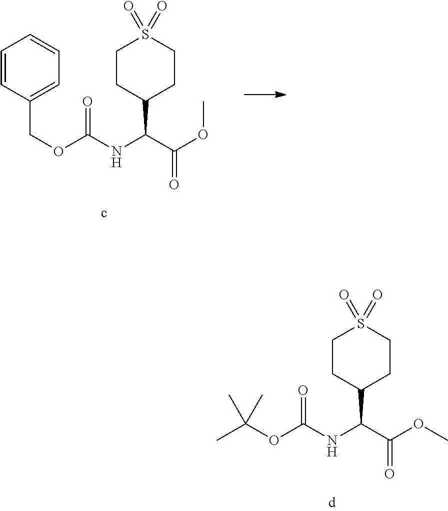

Z-protected amino ester c (804 mg, 2.27 mmol) was dissolved in methanol (16 mL). To this solution was added BOC-anhydride (1.5 g, 6.8 mmol), followed by 20% Pd(OH)₂.C (250 mg). All air was removed from the reaction flask by house vacuum, and the mixture was stirred vigorously for 5 min. The flask was then filled with hydrogen gas and allowed to stir vigorously at room temperature for 6 h. After evacuating the hydrogen atmosphere, the mixture was filtered through Celite using methanol, and crude product d was obtained by evaporation of the solvent (508 mg, 1.56 mmol, 70% yield).

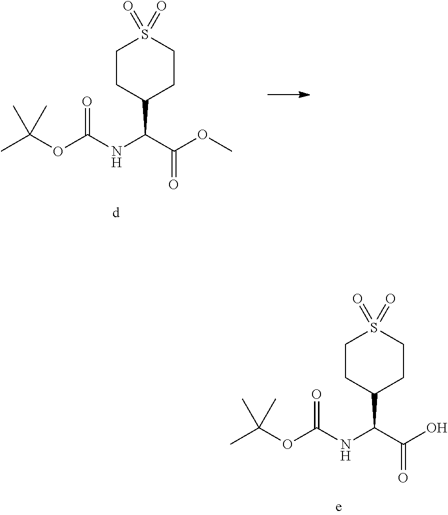

Ester d (508 mg, 1.56 mmol) was dissolved in 8 mL of THF. Deionized water (4 mL) was added, followed by LiOH.H₂O (120 mg, 2.8 mmol). The mixture was stirred at room temperature overnight, acidified using aqueous 1 N HCl and extracted into ethyl acetate (3×25 mL). The organic extracts were dried further with Na₂SO₄, filtered and concentrated to give 372 mg (1.21 mmol, 78% yield) of the N-Boc-protected cyclic sulfonyl amino acid e, which was carried on without purification.

### Example 10

### N-Boc-N-methyl-L-glycine

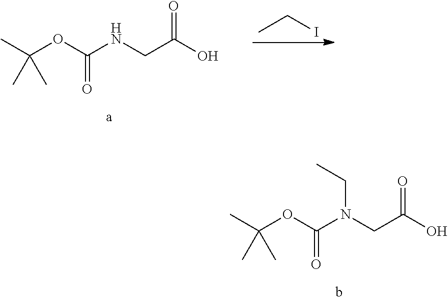

Following the general procedure of Grigg [Blaney, P.; Grigg, R.; Rankovic, Z.; Thornton-Pett, M.; Xu, J. Tetrahedron, 2002, 58, 1719-1737] a roundbottom flask was charged with sodium hydride (480 mg 60% dispersion in oil, 12.0 mmol, 4.0 equiv) and purged with nitrogen for 15 min THF (6.0 mL) was added to the flask, and the suspension was cooled to 0° C. using an ice water bath. A separate flask was charged with BOC-glycine a (525 mg, 3.0 mmol), dry THF (6.0 mL) and ethyl iodide (1.0 mL, 12 mmol, 4 equiv). This mixture was added dropwise to the NaH suspension in THF, with vigorous stirring at 0° C. After 1 h of stirring, the reaction was warmed to room temperature and allowed to stir overnight. The reaction was again cooled to 0° C., and methanol (4 mL) was added very slowly to quench the excess hydride. Deionized water was added to dilute the mixture, and methanol was removed under reduced pressure. Impurities were extracted into 90% ethyl acetate-hexanes, the aqueous layer was then acidified by adding solid citric acid until the pH reached 2-3. The product was extracted into 90% ethyl acetate-hexanes. This organic layer was dried (Na₂SO₄) and filtered. Removal of the solvents under reduced pressure afforded a quantitative yield of the product b.

### Example 11

### N-Boc-fluoro-L-alanine

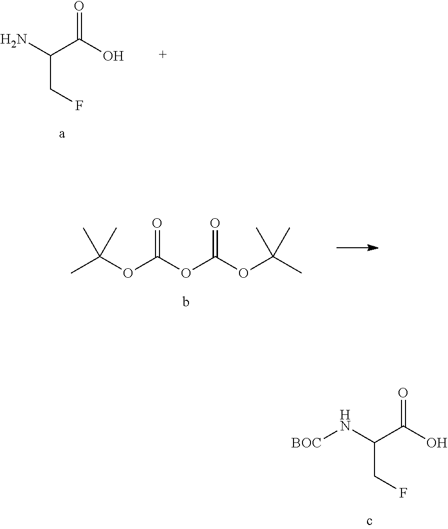

A mixture of unprotected amino acid a (775 mg, 7.24 mmol) and sodium carbonate (1.69 g, 16.0 mmol) was dissolved in a 1:1 solution of deionized water and THF (15 mL each). To this mixture was added BOC-anhydride b (1.73 g, 7.96 mmol). The mixture was stirred at room temperature overnight, and THF was removed under reduced pressure. The mixture was then acidified to pH 2-3 with saturated aqueous citric acid, and product was extracted into 10% ethyl acetate-dichloromethane. The organic layer was dried (Na₂SO₄), filtered and concentrated under reduced pressure to afford clean BOC-protected amino acid c (1.40 g, 6.7 mmol, 93%) to be used without further purification.

### Example 12

### (S)-tert-butyl 2-ethynylpyrrolidine-1-carboxylate

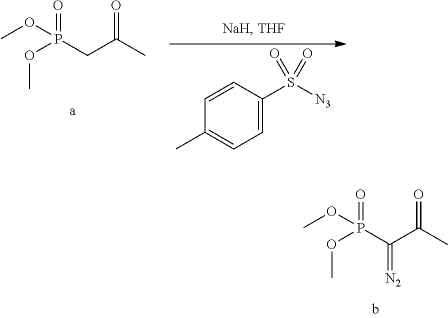

Dimethyl (2-oxopropyl)-phosphonate a (11.5 g, 69 mmol) was slowly added to a suspension of sodium hydride (3.3 g of a 60% suspension, 83 mmol) in 200 mL of anhydrous THF at 0° C. The mixture was stirred at 0° C. for 1 h. Then, toluenesulfonyl azide (15.0 g, 76 mmol), dissolved in 50 mL of THF, was added dropwise. The mixture was stirred for 30 min, filtered over celite and washed with ethyl acetate. The filtrate was concentrated, adsorbed on silica gel and purified by flash chromatography (SiO₂, 40% to 100% ethyl acetate/hexanes) to afford 8.5 g (64%) of compound b as a yellow oil.

MS: m/z=193 (M+H).

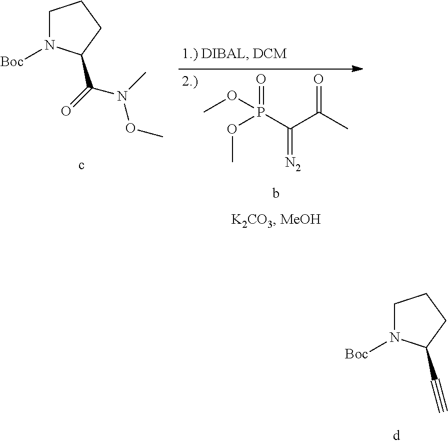

Following the general procedure of Hinkle (Hinkle, K. W. et al, Tet. Lett., 45, 2004, 5597-5599), Weinreb amide c (9.7 g, 37.6 mmol) was dissolved in 100 mL of DCM and the solution was cooled to −78° C. The DIBAL solution (94 mL of a 1M solution in heptane, 94 mmol) was slowly added over 20 min. After 20 min, the reaction was quenched by addition of 30 mL of dry methanol. Potassium carbonate (11.4 g, 82.7 mmol) was added, followed by reagent b (10.1 g, 52.6 mmol), dissolved in 50 mL of methanol. The mixture was stirred overnight while allowed to slowly warm up to room temperature. Rochelle's salt (sodium potassium tartrate) was added and the mixture was stirred for 8 h. The aqueous phase was then extracted with 3×100 mL of ether. The combined organic phases were dried with MgSO₄, filtered and concentrated. The crude oil was purified by flash chromatography (SiO₂, 0% to 30% ethyl acetate/hexanes) to afford 4.6 g (63%) of compound d (S)-tert-butyl 2-ethynylpyrrolidine-1-carboxylate as a colorless oil.

MS: m/z=196 (M+H).

### Example 13

### (S)-7-phenyl-2-(pyrrolidin-2-yl)-1H-pyrrolo[2,3-c]pyridine

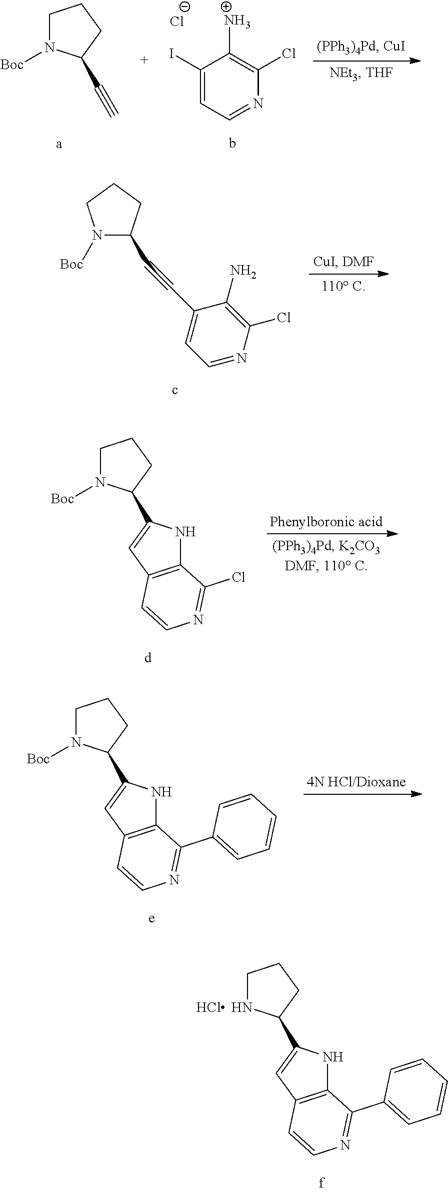

Compound b (2.3 g, 7.9 mmol), tetrakis(triphenylphosphine)palladium (590 mg, 0.5 mmol) and copper iodide (95 mg, 0.5 mmol) were suspended in 30 mL of dry THF. Triethylamine (5.6 mL, 40.8 mmol) was added, followed by alkyne a (2.0 g, 10.2 mmol), dissolved in 15 mL of THF. The mixture was stirred overnight at room temperature. The reaction mixture was filtered over celite and washed with ether. The filtrate was concentrated on silica gel and purified by flash chromatography (SiO₂, 0% to 60% ethyl acetate/hexanes) to afford 2.0 g (80%) of compound c as an orange oil.

MS: m/z=322 (M+H).

Compound c (2.0 g, 6.2 mmol) was dissolved in DMF (8 mL) and CuI (180 mg, 0.93 mmol) was added. The mixture was heated to 110° C. and stirred for 48 h. The mixture was cooled to room temperature, diluted with 40 mL of DCM and washed with 100 mL of water. The aqueous phase was extracted with 2×40 mL of DCM. The combined organic phases were washed with brine (100 mL), dried with MgSO₄, filtered and concentrated. The crude oil was adsorbed on silica gel and purified by flash chromatography (SiO₂, hexanes to ethyl acetate) to afford 740 mg (37%) of compound d as a yellow oil.

MS: m/z=322 (M+H).

Compound d (740 mg, 2.3 mmol), Phenylboronic acid (421 mg, 3.4 mmol), tetrakis(triphenylphosphine)palladium (213 mg, 0.18 mmol) and potassium carbonate (470 mg, 3.4 mmol) were suspended in DMF (8 mL). Deoxygenated water (0.7 mL) was added and nitrogen was bubbled through the mixture for 5 min. Then, the mixture was heated to 110° C. and stirred for 1 h. The solution was cooled down to room temperature, diluted with 30 mL of DCM and washed with water (50 mL). The aqueous phase was extracted with DCM (2×25 mL). The combined organic phases were washed with brine (50 mL), dried with MgSO₄, filtered and concentrated. The crude oil was adsorbed on silica gel and purified by flash chromatography (SiO₂, hexanes to ethyl acetate) to afford 660 mg (79%) of e as a yellow oil.

MS: m/z=364 (M+H).

Compound e (660 mg, 1.8 mmol) was dissolved in a 4N solution of HCl in 1,4-dioxane (45 mL, 180 mmol) and stirred at room temperature for 30 min. Then, the solution was concentrated to afford 539 mg (100%) of f (S)-7-phenyl-2-(pyrrolidin-2-yl)-1H-pyrrolo[2,3-c]pyridine as a pale yellow solid.

MS: m/z=264 (M+H).

### Example 14

### (S)-4-phenyl-6-(pyrrolidin-2-yl)-5H-pyrrolo[3,2-d]pyrimidine

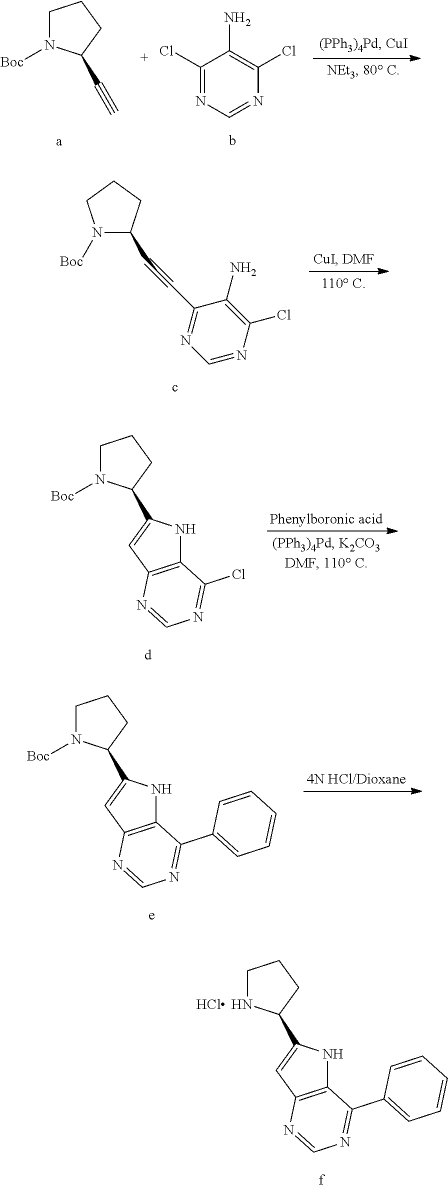

Compound a (450 mg, 2.3 mmol), 4,6-dichloro-5-aminopyrimidine b (1.1 g, 6.9 mmol), tetrakis(triphenylphosphine)palladium (133 mg, 0.11 mmol) and copper iodide (22 mg, 0.11 mmol) were dissolved in triethylamine (5 mL). The mixture was stirred at 80° C. for 2 h. The reaction mixture was then cooled down to room temperature, filtered over celite and washed with ether. The filtrated was concentrated on silica gel and purified by flash chromatography (SiO₂, 0% to 60% ethyl acetate/hexanes) to afford 452 mg (61%) of compound c as a yellow oil.

MS: m/z=323 (M+H).

Compound c (330 mg, 1.0 mmol) was dissolved in DMF (2 mL). Copper iodide (29 mg, 0.15 mmol) was added and the mixture was stirred at 110° C. for 2 h. The mixture was cooled down to room temperature, diluted with 20 mL of DCM, adsorbed on silica gel and purified by flash chromatography (SiO₂, hexanes to ethyl acetate) to afford 320 mg (97%) of compound d as a yellow oil.

MS: m/z=323 (M+H).

Compound d (320 mg, 1.0 mmol), phenylboronic acid, tetrakis(triphenylphosphine)-palladium and potassium carbonate were suspended in DMF. Deoxygenated water was added and nitrogen was bubbled through the mixture for 5 min. Then, the mixture was heated to 110° C. and stirred for 1 h. The solution was cooled down to room temperature, diluted with 30 mL of DCM and washed with water. The aqueous phase was extracted with DCM (2×). The combined organic phases were washed with brine, dried with MgSO₄, filtered and concentrated. The crude oil was adsorbed on silica gel and purified by flash chromatography (SiO₂, hexanes to ethyl acetate) to afford 351 mg (96%) of e as a yellow oil. MS: m/z=365 (M+H).

Compound e (350 mg, 0.96 mmol) was dissolved in a 4N solution of HCl in 1,4-dioxane and stirred at room temperature for 30 min. Then, the solution was concentrated to afford 281 mg (97%) of f (S)-4-phenyl-6-(pyrrolidin-2-yl)-5H-pyrrolo[3,2-d]pyrimidine as a pale yellow solid. MS: m/z=265 (M+H).

### Example 15

### (S)-2-methyl-4-phenyl-6-(pyrrolidin-2-yl)-5H-pyrrolo[3,2-d]pyrimidine

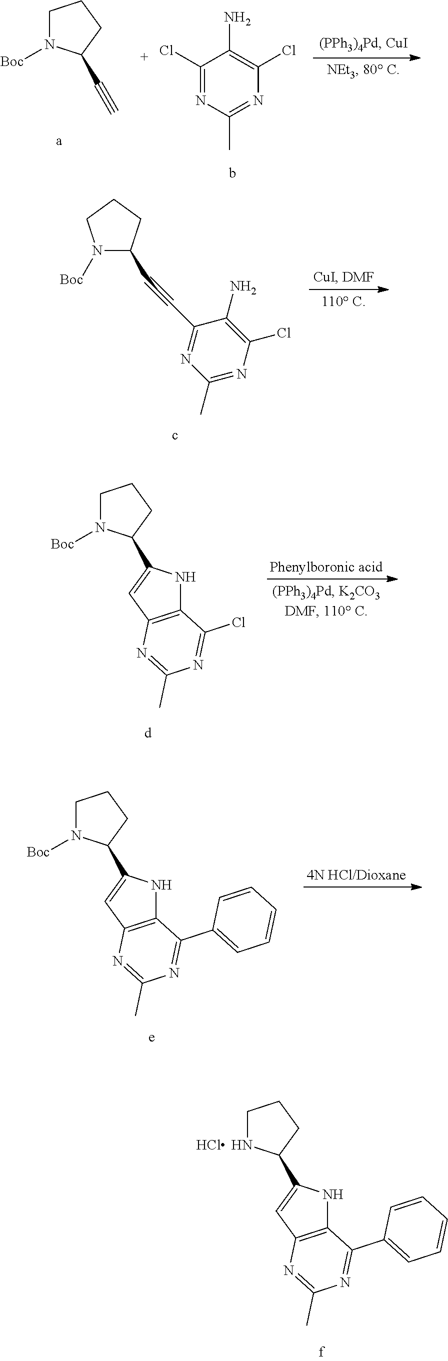

Compound a (450 mg, 2.3 mmol), 4,6-dichloro-5-aminopyrimidine b (3.2 g, 18.0 mmol), tetrakis(triphenylphosphine)palladium and copper iodide were dissolved in triethylamine. The mixture was stirred at 80° C. for 2 h. The reaction mixture was then cooled down to room temperature, filtered over celite and washed with ether. The filtrated was concentrated on silica gel and purified by flash chromatography (SiO₂, 0% to 60% ethyl acetate/hexanes) to afford 1.8 g (90%) of compound c as a yellow oil. MS: m/z=337 (M+H).

Compound c (1.8 g, 5.3 mmol) was dissolved in DMF. Copper iodide was added and the mixture was stirred at 110° C. for 2 h. The mixture was cooled down to room temperature, diluted with 20 mL of DCM, adsorbed on silica gel and purified by flash chromatography (SiO₂, hexanes to ethyl acetate) to afford 1.75 g (97%) of compound d as a yellow oil.

MS: m/z=337 (M+H).

Compound d (700 mg, 2.1 mmol), phenylboronic acid, tetrakis(triphenylphosphine)-palladium and potassium carbonate were suspended in DMF. Deoxygenated water was added and nitrogen was bubbled through the mixture for 5 min. Then, the mixture was heated to 110° C. and stirred for 1 h. The solution was cooled down to room temperature, diluted with DCM and washed with water (50 mL). The aqueous phase was extracted with DCM (2×). The combined organic phases were washed with brine, dried with MgSO₄, filtered and concentrated. The crude oil was adsorbed on silica gel and purified by flash chromatography (SiO₂, hexanes to ethyl acetate) to afford 785 mg (99%) of e as a yellow oil. MS: m/z=379 (M+H).

Compound e (785 mg, 2.1 mmol) was dissolved in a 4N solution of HCl in 1,4-dioxane and stirred at room temperature for 30 min. Then, the solution was concentrated to afford 540 mg (82%) of f (S)-2-methyl-4-phenyl-6-(pyrrolidin-2-yl)-5H-pyrrolo[3,2-d]pyrimidine as a pale yellow solid. MS: m/z=279 (M+H).

### Example 16

### IAP Inhibition Assays

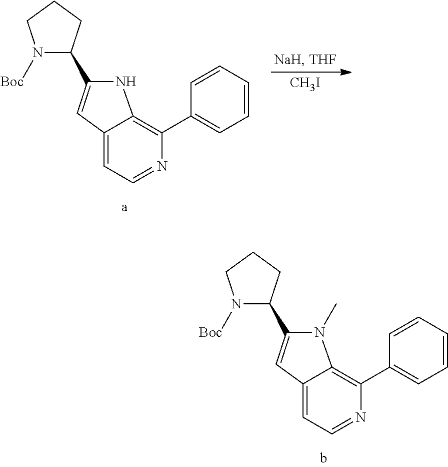

Compound a (40 mg, 0.11 mmol) was dissolved in THF (2.0 mL) and sodium hydride (7.0 mg of a 60% suspension in oil, 0.16 mmol) was added. The mixture was stirred at room temperature for 10 minutes and iodomethane (16 mg, 0.11 mmol) was added and the mixture was then stirred for 45 min. Then, the mixture was filtered over celite and washed with ethyl acetate (20 mL). The filtrate was concentrated on silica gel and purified by flash chromatography (100% Hex to 100% EtOAc) to afford 24 mg (58%) of compound bas a white solid. LCMS: m/z=378.

### Example 17

### IAP Inhibition Assays

In the following experiments was used a chimeric BIR domain referred to as MLXBIR3SG in which 11 of 110 residues correspond to those found in XIAP-BIR3, while the remainder correspond to ML-IAP-BIR. The chimeric protein MLXBIR3SG was shown to bind and inhibit caspase-9 significantly better than either of the native BIR domains, but bound Smac-based peptides and mature Smac with affinities similar to those of native ML-IAP-BIR. The improved caspase-9 inhibition of the chimeric BIR domain MLXBIR3SG has been correlated with increased inhibition of doxorubicin-induced apoptosis when transfected into MCF7 cells.

### MLXBIR3SG Sequence:

(SEQ ID NO.: 1)
MGSSHHHHHHSSGLVPRGSHMLETEEEEEEGAGATLSRGPAFPGMGSEEL
RLASFYDWPLTAEVPPELLAAAGFFHTGHQDKVRCFFCYGGLQSWKRGDD
PWTEHAKWFPGCQFLLRSKGQEYINNIHLTHSL

Similar cIAP1-BIR3/XIAP-BIR3 and cIAP2-BIR3/XIAP-BIR3 chimeric proteins, cIAP1XBIR3 and cIAP2XBIR3, respectively, were engineered, expressed, and purified in a similar fashion to MLXBIR3SG. As with MLXBIR3SG, the amino acid sequences of these BIR domains retain the native cIAP peptide-binding sites.

cIAP1XBIR3 Sequence:

(SEQ ID NO.: 2)
MQTHAARMRTFMYWPSSVPVQPEQLAAAGFYYVGRNDDVKCFSCDGGLRC
WESGDDPWVEHAKWFPGCEFLIRMKGQEYINNIHLTHSL

cIAP2XBIR3 Sequence:

(SEQ ID NO.: 3)
MQTHAARFKTFFNWPSSVLVNPEQLAAAGFYYVGNSDDVKCFSCDGGLRC
WESGDDPWVQHAKWFPGCEYLIRIKGQEYINNIHLTHSL

### Fluorescence Polarization Peptide Binding Assay

Polarization experiments were performed on an Analyst HT 96-384 (Molecular Devices Corp.) according to the procedure of Keating, S. M., Marsters, J, Beresini, M., Ladner, C., Zioncheck, K., Clark, K., Arellano, F., and Bodary, S. (2000) in Proceedings of SPIE: In Vitro Diagnostic Instrumentation (Cohn, G. E., Ed.) pp 128-137, Bellingham, Wash. Initial polarization experiments were performed in order to determine dissociation constants (K_{d}) between IAP protein BIR domains and fluorescent probes. Samples for fluorescence polarization affinity measurements were prepared by addition of serial dilutions of MLXBIR3SG, XIAP-BIR3, cIAP1XBIR3, or cIAP2XBIR3 in polarization buffer (50 mM Tris [pH 7.2], 120 mM NaCl, 1% bovine globulins, 5 mM DTT and 0.05% octylglucoside) to 5 nM 5-carboxyflourescein (5-FAM)-conjugated AVP-diphenylalanine-AKK (AVP-diPhe-FAM). The resulting reactions were incubated for 30 minutes at room temperature in 384-well black HE96 plates (Molecular Devices Corp.; Sunnyvale, Calif.) and fluorescence values were measured using standard filters for the fluorescein fluorophore (λₑₓ=485 nm, λₑₘ=530 nm). Fluorescence polarization values were plotted as a function of the protein concentration and the 50% effective concentration (EC₅₀) values were obtained by fitting the data to a four-parameter equation using KaleidaGraph software (Synergy Software; Reading, Pa.). The apparent K_{d} values were determined from the EC₅₀ values.

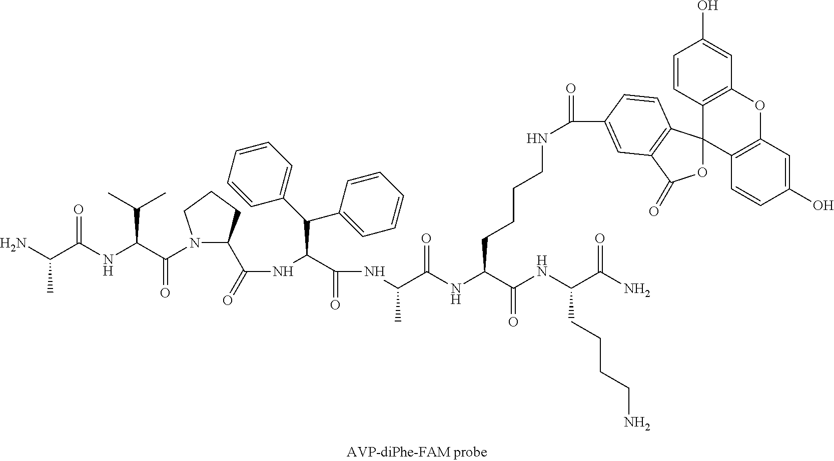

Inhibition constants (Kᵢ) for the antagonists were determined by addition of 0.06 μM MLXBIR3SG, 0.5 μM XIAP-BIR3, 0.2 μM cIAP1XBIR3 or 0.4 μM cIAP2XBIR3 to wells containing 1:3 serial dilutions of the antagonists, and 5 nM AVP-diPhe-FAM probe in the polarization buffer. After a 10 to 30-minute incubation period, the fluorescence value of each sample was measured. Fluorescence polarization values were plotted as a function of the antagonist concentration, and the 50% inhibitory concentration (IC₅₀) values were determined by fitting the data to a four-parameter equation using KaleidaGraph software. The Kᵢ values for the antagonists were determined from the IC₅₀ values (Keating et al. 2000). Compounds of the invention that were tested in this assay exhibited Kᵢ and IC₅₀ values shown in table 1 (all values nM).

TABLE 1

| | MLXBIR3SG | | cIAP1XBIR3 | | cIAP2XBIR3 | | XIAPBIR3 | |
|---|---|---|---|---|---|---|---|---|
| cmpd | Kᵢ | IC₅₀ | Kᵢ | IC₅₀ | Kᵢ | IC₅₀ | Kᵢ | IC₅₀ |
| 1 | 36.0 | 158.0 | 71.3 | 460.5 | 108.6 | 502.4 | 90.5 | 508.3 |
| 3 | 29.8 | 130.8 | | | | | 79.2 | 445.1 |
| 4 | 35.6 | 156.3 | | | | | 76.9 | 434.2 |
| 5 | 31.7 | 139.3 | | | | | 91.5 | 514.2 |
| 6 | 81.9 | 359.2 | | | | | 201.2 | 1130.3 |
| 7 | 25.7 | 112.7 | | | | | 85.6 | 480.8 |
| 8 | 24.6 | 107.8 | | | | | 89.0 | 500.2 |
| 9 | 14.1 | 62.0 | | | | | 64.9 | 364.4 |
| 10 | 22.6 | 99.3 | | | | | 102.2 | 574.1 |
| 11 | 147.8 | 648.3 | 30.1 | 194.4 | | | 121.3 | 681.8 |
| 12 | 32.9 | 144.3 | | | | | 64.4 | |
| 13 | 20.7 | 91.0 | | | | | 63.8 | |

We claim:

1. A compound of formula (I) 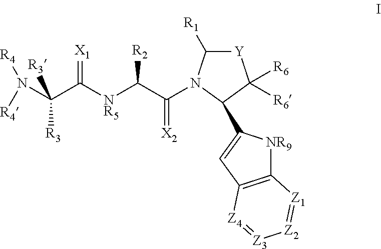
wherein
X₁ and X₂ are each independently O or S;
Y is a bond, (CR₇R₇)ₘ, O or S;
Z₁, Z₂, Z₃ and Z₄ are each independently N or CQ;
Q is H, halogen, hydroxyl, carboxyl, amino, nitro, cyano, alkyl, a carbocycle or a heterocycle; wherein said alkyl, carbocycle and heterocycle is optionally substituted with one or more hydroxyl, alkoxy, acyl, halogen, mercapto, oxo, carboxyl, acyl, optionally substituted alkyl, amino, cyano, nitro, amidino, guanidino an optionally substituted carbocycle or an optionally substituted heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)—;
R₁ is H, OH or alkyl; or R₁ and R₂ together form a 5-8 member heterocycle;
R₂ is alkyl, a carbocycle, carbocyclylalkyl, a heterocycle or heterocyclylalkyl each optionally substituted with halogen, hydroxyl, oxo, thione, mercapto, carboxyl, alkyl, haloalkyl, acyl, alkoxy, alkylthio, sulfonyl, amino and nitro, wherein said alkyl, acyl, alkoxy, alkylthio and sulfonyl are optionally substituted with hydroxy, mercapto, halogen, amino, alkoxy, hydroxyalkoxy and alkoxyalkoxy;
R₃ is H or alkyl optionally substituted with halogen or hydroxyl; or R₃ and R₄ together form a 3-6 heterocycle;
R₃′ is H, or R₃ and R₃′ together form a 3-6 carbocycle;
R₄ and R₄′ are independently H, hydroxyl, amino, alkyl, carbocycle, carbocycloalkyl, carbocycloalkyloxy, carbocycloalkyloxycarbonyl, heterocycle, heterocycloalkyl, heterocycloalkyloxy or heterocycloalkyloxycarbonyl; wherein each alkyl, carbocycloalkyl, carbocycloalkyloxy, carbocycloalkyloxycarbonyl, heterocycle, heterocycloalkyl, heterocycloalkyloxy and heterocycloalkyloxycarbonyl is optionally substituted with halogen, hydroxyl, mercapto, carboxyl, alkyl, alkoxy, amino, imino and nitro; or R₄ and R₄′ together form a heterocycle;
R₅ is H or alkyl;
R₆, and R₆′ are each independently H, alkyl, aryl or aralkyl;
R₇ is H, cyano, hydroxyl, mercapto, halogen, nitro, carboxyl, amidino, guanidino, alkyl, a carbocycle, a heterocycle or —U-V; wherein U is —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)— and V is alkyl, a carbocycle or a heterocycle; and wherein one or more CH₂ or CH groups of an alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —C(O)—O— or —O—C(O)—; and an alkyl, carbocycle and heterocycle is optionally substituted with hydroxyl, alkoxy, acyl, halogen, mercapto, oxo, carboxyl, acyl, halo-substituted alkyl, amino, cyano nitro, amidino, guanidino an optionally substituted carbocycle or an optionally substituted heterocycle;
R₈ is H, alkyl, a carbocycle or a heterocycle wherein one or more CH₂ or CH groups of said alkyl is optionally replaced with —O—, —S—, —S(O)—, S(O)₂, —N(R₈), or —C(O)—; and said alkyl, carbocycle and heterocycle is optionally substituted with hydroxyl, alkoxy, acyl, halogen, mercapto, oxo (═O), carboxyl, acyl, halo-substituted alkyl, amino, cyano nitro, amidino, guanidino an optionally substituted carbocycle or an optionally substituted heterocycle;
R₉ is H, alkyl or acyl; and
m is 0 to 4;
provided that the compound is other than: 2-[(2R)-1-[N-methyl-L-alanyl-(2S)-2-cyclohexylglycyl]-2-pyrrolidinyl]-1H-indole.

2. The compound of claim 1, wherein Z₁, Z₃ and Z₄ are each independently CQ and Z₂ is N.

3. The compound of claim 1, wherein In an embodiment Z₁ and Z₃ are each independently CQ and Z₂ and Z₄ are each N.

4. The compound of claim 1, wherein Q is a carbocycle or heterocycle optionally substituted with alkyl, a carbocycle or a heterocycle; wherein any alkyl, carbocycle or heterocycle is optionally substituted with halogen, amino, hydroxyl, mercapto, carboxyl, alkoxy, alkoxyalkoxy, hydroxyalkoxy, alkylthio, acyloxy, acyloxyalkoxy, alkylsulfonyl, alkylsulfonylalkyl, alkylsulfinyl, and alkylsulfinylalkyl; and wherein one or more CH₂ or CH groups of any foregoing alkyl is optionally replaced with —O—, —S—, —S(O)—, S(O)₂, —N(R₈)—, —C(O)—, —C(O)—NR₈—, —NR₈—C(O)—, —SO₂—NR₈—, —NR₈—SO₂—, —NR₈—C(O)—NR₈—, —NR₈—C(NH)—NR₈—, —NR₈—C(NH)—, —C(O)—O— or —O—C(O)—;

5. The compound of claim 1, wherein Q is a carbocycle or heterocycle selected from the group consisting of IIIa-IIIs: 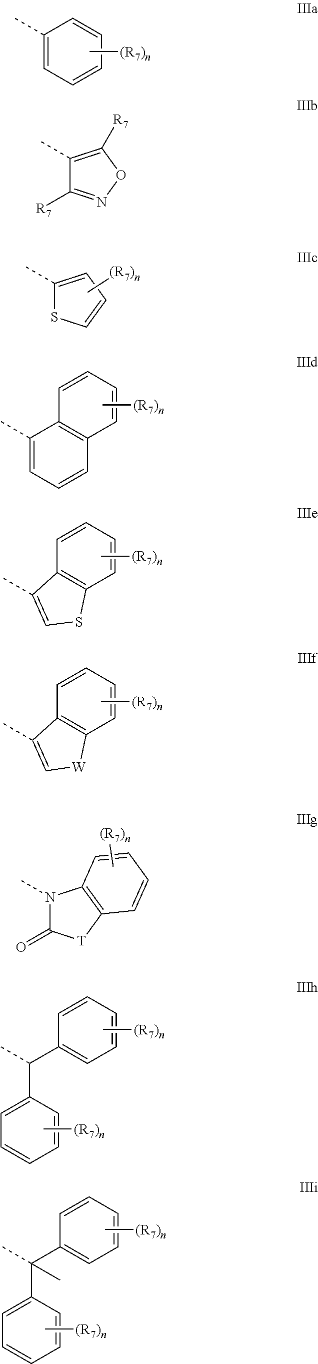 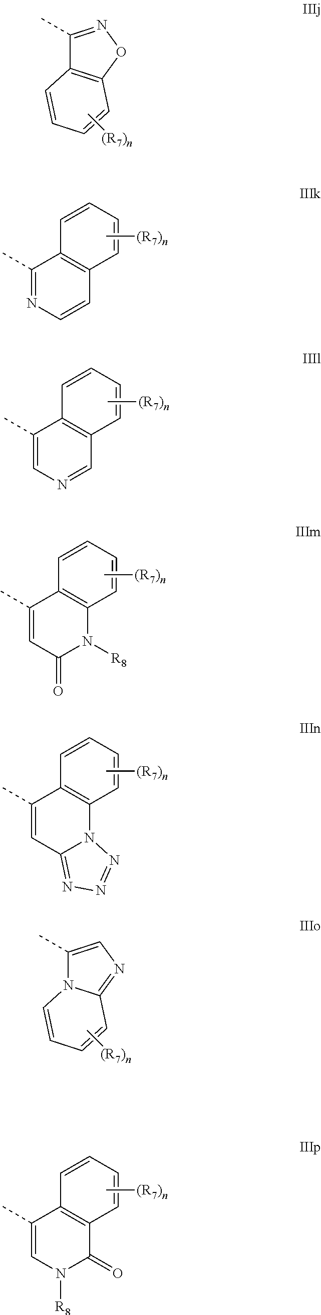 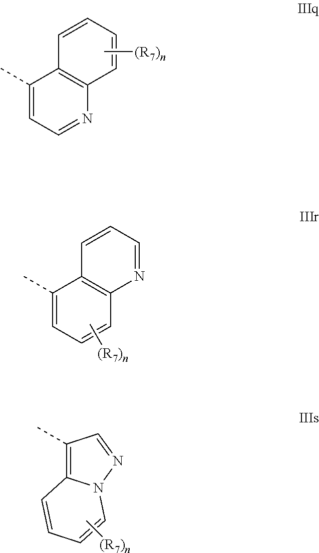
wherein n is 1-4; T is O, S, NR₈ or CR₇R₇; and W is O, NR₈ or CR₇R₇.

6. The compound of claim 1, wherein R₁ is H.

7. The compound of claim 1, wherein R₂ is alkyl, cycloalkyl or a heterocycle.

8. The compound of claim 1, wherein R₂ is selected from the group consisting of t-butyl, isopropyl, cyclohexyl, tetrahydropyran-4-yl, N-methylsulfonylpiperidin-4-yl, tetrahydrothiopyran-4-yl, tetrahydrothiopyran-4-yl (in which the S is in oxidized form SO or SO₂), cyclohexan-4-one, 4-hydroxycyclohexane, 4-hydroxy-4-methylcyclohexane, 1-methyl-tetrahydropyran-4-yl, 2-hydroxyprop-2-yl, but-2-yl, thiophen-3-yl, piperidin-4-yl, N-acetylpiperidin-4-yl, N-hydroxyethylpiperidin-4-yl, N-(2-hydroxyacetyl)piperidin-4-yl, N-(2-methoxyacetyl)piperidin-4-yl, pyridin-3-yl, phenyl and 1-hydroxyeth-1-yl.

9. The compound of claim 1, wherein R₃ is methyl.

10. The compound of claim 1, wherein R₄ is H or methyl, and R₄′ is H.

11. The compound of claim 1, wherein R₅ is H.

12. The compound of claim 1, wherein R₆ and R₆′ are both H.

13. The compound of claim 1, wherein X₁ and X₂ are both O.

14. The compound of claim 2, wherein R₁ is H; R₂ is isopropyl, t-butyl, cyclohexyl or pyran; R₃ is methyl; R_{3′} is H; R₄ is methyl, R₄′ is H; R₅ is H; X₁ and X₂ are both O; and R₆ and R_{6′} are both H.

15. A method of inducing apoptosis in a cell comprising introducing into said cell a compound of claim 1.

16. A method of sensitizing a cell to an apoptotic signal comprising introducing into said cell a compound of claim 1.

17. The method of claim 16, wherein said apoptotic signal is induced by contacting said cell with a compound selected from the group consisting of cytarabine, fludarabine, 5-fluoro-2′-deoxyuridine, gemcitabine, methotrexate, bleomycin, cisplatin, cyclophosphamide, adriamycin (doxorubicin), mitoxantrone, camptothecin, topotecan, colcemid, colchicine, paclitaxel, vinblastine, vincristine, tamoxifen, finasteride, taxotere and mitomycin C or radiation.

18. The method of claim 16, wherein said apoptotic signal is induced by contacting said cell with Apo2L/TRAIL.

19. A method for inhibiting the binding of an IAP protein to a caspase protein comprising contacting said IAP protein with a compound of claim 1.

20. A method for treating a disease or condition associated with the overexpression of an IAP in a mammal, comprising administering to said mammal an effective amount of a compound of claim 1.
